SEQUENCE LISTING (20 sequences)

SEQ ID NO: 1 - DNA, 25 bp, Artificial Sequence
  Primer E1-1200F
agtgacacacaggtgttcaaagacg

SEQ ID NO: 2 - DNA, 20 bp, Artificial Sequence
  Primer E1-1660R
gcaagtcctccagcgttctg

SEQ ID NO: 3 - DNA, 21 bp, Artificial Sequence
  Primer E2-13291F
ccacaaacacagggcagtaag

SEQ ID NO: 4 - DNA, 22 bp, Artificial Sequence
  Primer E3-18487R
ataaccacatcaaataagcgtg

SEQ ID NO: 5 - DNA, 20 bp, Artificial Sequence
  Primer E3-18011F
atcatcctatttctatgggc

SEQ ID NO: 6 - DNA, 21 bp, Artificial Sequence
  Primer E3-18487R
tcaacatagcaccacagtagg

SEQ ID NO: 7 - DNA, 18 bp, Artificial Sequence
  Primer E4-19068F
ttggtattggcctgatgg

SEQ ID NO: 8 - DNA, 25 bp, Artificial Sequence
  Primer E4-19862R
acataagtctcttctgatgctattc

SEQ ID NO: 9 - DNA, 26 bp, Artificial Sequence
  Primer E5/6-21009F
ggcccctaatttacaaagtgccattg

SEQ ID NO: 10 - DNA, 18 bp, Artificial Sequence
  Primer E5/6-21727R
atgtgtttcctgggaatg

SEQ ID NO: 11 - DNA, 28 bp, Artificial Sequence
  Primer E7-22366F
tcactagaacacttgacttagtagtacg

SEQ ID NO: 12 - DNA, 18 bp, Artificial Sequence
  Primer E7-23075R
gcacccacaaacacaagc

SEQ ID NO: 13 - DNA, 18 bp, Artificial Sequence
  Primer I10-6995
agcaagaaatacaccgag

SEQ ID NO: 14 - DNA, 20 bp, Artificial Sequence
  Primer I11R.7343
agcaggtagaattgtatgag

SEQ ID NO: 15 - DNA, 22 bp, Artificial Sequence
  Primer E9-24349F
ttagatttgtgggtcattgatc

SEQ ID NO: 16 - DNA, 21 bp, Artificial Sequence
  Primer E9-25143R
aaatatgttggctcttcagcg

SEQ ID NO: 17 - DNA, 18 bp, Artificial Sequence
  Primer M13F
tgtaaaacgacggccagt

SEQ ID NO: 18 - DNA, 18 bp, Artificial Sequence
  Primer M13R
caggaaacagctatgacc

SEQ ID NO: 19 - DNA, 28339 bp, Homo sapiens
  misc_feature (1)...(28339): TNFRSF6
tattagatgctcagagtgtgtgcacaaggctggcacgcccagggtcttcctcatggcactaacagtctactgaaaggtggaacagagacaagcctatcaacacctacaagactggtggtaagtgcagtgacagatgcaaaacacagggtgatggaaagccctcaggagggtaacctaacctagatttgagggcccaaacaggctccagaagaaaatgtcaactgagaggaagcctgaaggatgaacagtgggctaagcaaagggttattaatgtgttattaatgggttgaatctaattgggaagggagagaggttgcagagtgaggtgcagagcttggtggacgatgccaaaggaatactgaaacctttagtgtgtccagtctggaactgcatccaaattcaggttcagtaatgatgtcattatccaaacataccttctgtaaaattcatgctaaactacctaagagctatctaccgttccaaagcaatagtgactttgaacagtgttcaccagagcacgaaagaattacaagatttttttttaaagaaaattggccaggaaataatgagtaacgaaggacaggaagtaattgtgaatgtttaatatagctggggctatgcgatttggcttaagttgttagctttgttttcctcttgagaaataaaaactaaggggccctcccttttcagagccctatggcgcaacatctgtactttttcatatggttaactgtccattccagaaacgtctgtgagcctctcatgttgcagccacaacatggacagcccagtcaaatgccccgcaagtctttctctgagtgactccagcaattagccaaggctcctgtacccaggcaggacctctgcgctctgagctccattctccttcaagacctccccaacttcccaggttgaactacagcagaagcctttagaaagggcaggaggccggctctcgaggtcctcacctgaagtgagcatgccagccactgcaggaacgccccgggacaggaatgcccatttgtgcaacgaaccctgactccttcctcaccctgacttctccccctccctacccgcgcgcaggccaagttgctgaatcaatggagccctccccaacccgggcgttccccagcgaggcttccttcccatcctcctgaccaccggggcttttcgtgagctcgtctctgatctcgcgcaagagtgacacacaggtgttcaaagacgcttctggggagtgagggaagcggtttacgagtgacttggctggagcctcaggggcgggcactggcacggaacacaccctgaggccagccctggctgcccaggcggagctgcctcttctcccgcgggttggtggacccgctcagtacggagttggggaagctctttcacttcggaggattgctcaacaaccatgctgggcatctggaccctcctacctctggtgagccctctcctgcccgggtggaggcttaccccgtcttagtcccggggataggcaaagtggggcgggcgcgggacgcgtgcgggattgcggcggcagcggcgcacgcgggcacctgggagcggcgggctgctgcgggaggcgttggagactggctcccgggggctgttaggaccttccctcaggcccgggtgctcagaacgctggaggacttgcttttcttgggccttgatgcgaagtgctgaccccgctgggcaggcggggcagctccggcgctcctcggagaccactgcgctccacgttgaggtgggcgtggggtgcggacaggaattgaagcggaagtctgggaagctttagggtcgctggagggggaccccggttggagagaggagcggaactcctggacaagccctgacaagccaagccaaaggtccgctccggcgcgggtgggtgagtgcgcgccgccccgcgggggcggggagagagcctgcagccttcagaacagatattgctcattttctggcagttctcagacgtaggaaataagtcagcaccgaagcagtggttaagccggagggctcggaagaacggcaccttttctttctcgaaaaagttatatgggggctgaatgagcttctggaggcttgtttaccgttttttattgtcacacagaaaaagaaactgccttgtctcccttccgggaattctctctttaagactgtaagtcgctgcctgagtggtttcattttgttttgtttttctgcccttctctttcttcttttgccctttcttagcttgcactcccatggtgatttctgcttggtctcctgctggggttggtggtactcgttcccaccgcacagaacccggcgcctattattggccaagaaacttgagcagcctgttttgaaaagtccctcgctcagaaatgccagcttgcagatggctaatcaaagagacgtggatccaggaggctcatttgagtaccggagcctctgagagtccatggatttttattttattgggccccaacaaagtgccaggcatagcgagagaagtgtttacaaatgtgatctccgcggattctcacgagagccatgtagtggggaagataaccacatccccattttacagatggcgaaatgaggttcagagagcttcagtaattcagccaagatcacacagctagtaaatgggagatatgggattgaactcagatctaattccaaaactcaggctgttcgctgattgcatctgatatccagattacaccaagaggctgagatgtctgggtccattttatgattagaaaaaaaaatcctgttcctttcagaaatagtgtgcactatttgggattctttagaaacttccttggagaaaattaatgattcaagattgagaaaacatttatacaacctcaggccacactcttctctctcttgttattaaccattgcaccactgcaatgttggctgctttgaaaatctcatagtttttggacagcagtattagtaaagtgtaagaaaattgtcaaagcttggagctatgcttgttgaacttttgtaccaatagcacctttaccagaggctgcgtgtttaaagcccgaagaaaactttgcttttctgaaagaatacacacacacacgcatatgtaaatattcatacatttatgtatatatacatattataatacctataagttaggtataacttatatttgtatatgatatatggcctaggaaattaaggcttattaaataaaatttataaatgcagatgagtcaaatacaaagatcagacataactctatcacctaagtaatcattgtttacgttttgcagtttatcttccatttctcccctctaatatgactcttttaaatttagttacacagaaattggttttgcgctcacgacatgcctaacatcaggcctttatccttaaagaagtttcaacactcttgtgtcacctcatgtgtctacagaaaatgtttgcttttatgccttcacggttatgttttcttagtagtagcaataaataaaatagatgcaaagtgctaattacttggaatacagaagcaaattttaattctattaatgggctggacatagatgggtggggagctgttttctgcttttcccctcttcccctagtcctctgactctggtgataactatagtcaacaagtatccccatccttaaccccttttgcttctctttctctttccttctctcaaccttcctggtgttagttttctctttcctatttctgagtgtttttactccgacacttacaataaaaaagtttgaaatcccaaaaagttgacaaatttctaaaggagacagaaaaagagtcaaacttactttacaaattaatttgagaaatagctgtgggttgcattttttttttttattgtgcatgaccctgttattggccaagaaacttgagcagcctgttttggaaagtccctcgctcagaaatgccagcttgctaattgaagggatgtggatccatgaggctcatttgaattccgaagcctctaagagtccatggatttttattttattgggccccaacaggttttgtcttgcattatttaggacattggccaggcaatcactggacagccagtttgctctcctttacattttgtagaatggttatcttatggtcaaaggaaagggagagggcctttggaaaataaagctgtgtatttacaagcagtcatacagaatagttttgtggggaaaataaagaacagttatttaatttgtaatttaatcatttttcttaaaccaacccaaagagcataaaatggaatatttatttatttttgtagcttaatttgtaatttaataatttttcttaagccaacctaacgaacataaattgggctgtttattttcatttttaatgggccatatttacatcacaaaggacttgagaagtcttacaaatacaataaaatgataaaatatgaattaagacattagcaaccaggatggaaaaacacaaatttgtaaatgatggccaggtttgcaagcaaaaattaaattgataaatggcagcacccccactaccagctacagttagactaatgaaaaatcctctaatgttcatgcctgaaatacagttaccacccttagcagcaaattagaggcaaagaagttctggaatttggacgttgtctctgagtaagctctttgagacactggacaagtaatttccttctgaatactcttatactgacacataggagaagtgtcagtgtttcaaaggaatctctttgaaggctactacattgctttaggggtgccagattaccagagtaaactgccagtttgagagaataactgtttcttaagagatggtctgaaacctaagtaaggaaatccccccacttgaaagcccctaggaagttaattgcttttggcaaattatcttgggccccaaaagtcccttgatgagcacatgttttgtaataggtactaaagactatttacttatgatgaaaaataaaataacacaatttaaaacttgaaattgataaaaagcaacttgaaaccaaatttaggacagtgaacccatccctttcttatttttaaataggtaaatatagaaaaaatgaccacatgatgtgaaacaaaaagcaaatatttaaaaactacatttatactttatctcaccacactaatgtaaagaattttgattttacttataaattcttagccttgtctacacatatacataattgcatgtttaatttgagctatatatagtctgttttttgttcctttgcatgttacacaaacctttttctatttctacataatttttatctcatattttttattggatacattatagagagagcatttattaaatgagctgggattactataatttttattatttattttttaatttattacttatttttataatctactattgaaaaaaagcatttcttaaatgtactgggatacatatatatatatgactttttcttgttactaaaaaattactcatagaacttacatattaaaattttaggacttgtgatgaaaaataataaaataatttaaaacgataaaaagcaacataattttatatgttgtaccttgtacttccttataaggtacaacactacaatagccttagtgtgtagttttctttttgctatgtatgtatatatatatattcataaaagtggaaaaataacatttaataaatcctctttattttctgggaaatccttgctttaaaaaaagtctttagctttttatatagttcgacttctaacaccaattaaatatcttcaattcagtctcaagcaggaacaaatcttcagtccacttgcctacctttactgtctttgaagtgacagtgtgagtagaaaagcccaaatcagagagaaattgacttttgtaagattggtaacttcaccttccccggaaggaaatgaggtggttcttccctttgatcacaactgctttcttgatttgaaataaactgttgtacagcccagtcaccccatctttagacatttagaaatggcaatcatagttatataggacaactagcctgagtcccactaagtagaaatgatagtgaatttagtataagattatacctttaggctgggcaggtgactcacacctgtcacccagcacttcaggaggccaaggtgggtggatcatggatcacttgagattaggagtttgagaccagcctggccaacatggcaaaaacccaactctactgaaaatacacaaattagctgggtgtggtggcgtgcctgttatctcagctactcgggaggtcgcggcaggagaatcactggaacctgggaggtggaagttgcagtgagccaagatcatgccactgcactccaggctgggcgacagagtgagactccgtctacaaagaaaacaaagaaacaaaaaaatcatacctttagagcaaggccagccttgtgaattagcaaatctgactacaaattcaatcagcagatgtactcagcccagcaaagtgagcaagcttactataggttgtgttaaggcatggactgggataaactggcaaaattagagtaaatttgacaatttgacattggattcattaatgatagcctgtccttcaatgaagtcaggcagcattgtgtttctattcacctctgtgttttgtgaggctataaaaatataagatatgagaaatgtgaatactttttatttgtgagggcagatatcttgaaaaagagagcgagaggaagtcagagtatgcgggtgaagactcttgttttttttgctgaggatttagctttggtttttagccattctcacagagatcattggtcctaattaactggttgttgttgtttttttttcatgtgaatgtctgtaaggttttaggcttgattttaaaaattgggcctatactcacagaaagacaaatactgcatgatctcgtttatatgtggaatctaaaaggtttgaatccataaaagtagagagtagaatagtggttaccagaggctagttgggggcaaagttgggggatttagggagatattggtcaaaggatacaaaatttcagttaggaggaataagttcaagagatctagtgtacaacttgatgtctatagctaataacaatgtattgtatacttgaaaattactaagagtagattttaagtgttctcgccacacacacacaaaagaaaatgataagtatgtgaaggaatgcccaatgtaattagctcgatttagctaattatcttccaaaatatgtacataattatcttccaaaatatgtacataattcaagacatcatgttatataccataaatatatacaatttttttatttgtcgaataaaaataatacaaaaaaaccctgggcctatactgcccacaggtagtggccacaggcagagctggtcccaagcccctccctctctctgtagtcccttatcccaggttgctttattcttgtatgacccacgtggcccctctgggcttgtaagggctctggggctgtattttaggactctatagcccttacattacccatttcctctaccaaaggtcatttctcttatcttcaagcttatttttggaagggtcattatttcttcatgagaagtcaaattctgatttgcaagtgctgaacacagatgccattagtctataaaactcttatattctcacatgtaaatgtagtaacctgctaacctcagattctgcttcctctaacctagttgccctgcaactgtgatgaacctcaaaatcaccaagggtacttccccattctcagagatcctgattccttaggtctatgtaggggccttagcactttttattaaataatgcatcactacaggattctgatgtctgtcggggtcttgagaactatttttctgactctagcactaaactcaaaatgtaatttcagatggaatttcctaggctcattcattttattttatttatggcttggccagtcatggatctaatcagtaacttgcaactgttgcttcaactacctctgtcctggactttccagagattggctttaagaaagaaagaggacatccctgggataagattaccatgtgtatttttatgtacacattttgaaaaacatacaaatgaatgggatctttgcttatctcttccatgtagaaaaatttattttacttctatttcaggttttcaataatgtagtaggacaagatgacatcactatatccctgttttttacacagatatattagagatgcattatcttattggaaattttaaactattcaataataaactattagtcttttagaatttctttttcctcaagtgttgataagtaactgaaagtctacaagcagacaagcagaattagtcggtggaacttcatttcttgctccatttttgtcctcagcagtagagggtggtgagatttcaacccagcctagcaggaccaataagtctggaaagaagtagctctagttcatattggcacctgctaattctagttctgaagaccatgggaaatctcttgcctatttagctcagtgatcatccaagatgggaaattcttgggctcagctgtgagggatcccaagtttatatgtaacactaatgtctcacaaatcaagaatccaagagaatcaagagactatagttcattccttatattaaattcttaggactgccaactgggtggctacagttttgggggctagaagtctgatatcaaggtgccaactggactggttccttctgaggaccacgagggcaggatctgttcaggcttgtagatggccttcttttccatgtgtcttcacattatcttcctctgtttgtatgtgtgtccaaatttcctcttctttttttttaatttagaaatttatactgtctacaagctttatatagctttagttaaaaaaaagcaaaaaaaaaaaaaaatcaaaacaaaaacagtgaagccaagacactcttccaaagtctggacccttccggccttccaaatacaagagctctggaagttgtatataccaaatttcctcttcttaaaatgtcaccagtcacattggattaaggcttaccctattaacaccattttaacttaatcacctctgtaaagaccctatctcctgatacagtcacattcggatgctcaggaagttagggtatcagtatcactatatgaatttgggagttgggggaggacacagtttagcccatgacagtcctgaatctcctttgctagatgcatgactgtgggccagtcacttcaccagttgggatactgccatctctatcagaaaaggggtttgggctaggtcatctataaagtcctttctgctctaaattgtaaatataatgagataccaacatttagtttaattgtttaaaattttagaagctaactggatgctttgctggtgtcaacactcccctttacaaaaaaaaattcggccgggcatggtggctcacgcctgtaatcccaacactttgggaggccgaggcaggcggatcacggggtcgagagattgagaccatctggccaacatggtgaaaccccgtctctactaaaaacacaaaaattagctgggcctggtggtgcacatctgtagtcccagctactcgggaagctgaggcaggagagtcacttgaacccgggaggcggagcttgcagtgagccgagatcgcgccactgcacctccagcctggcaacagagcgagactccgtctcaaaaaaataaataaataaataaaataaaataaaataaaataaaatctcactttctgtttctactgttctctcaagtacccctctgccttctgttacttctttgctgcattctgagaggaatggggtggggagcactctttcctgtagaaccccataacccatctattttcatcaatttatttgaaattcaaagccaggggttaaaatgtccactttcaatttgagatgtctaactttcagataaatctattaacatgcatttctaagtgacttttaagctaagcattttattttattgagctcgtatggaagggtaactattatacttgtataataaagtgcactttcaaaaatactcggttcaaatgtaaaataatattcgtaatattttaagacaaacctcctagaacttatatgtgtctttcttcccagagcagtctctatgggtgcctaaacaatactgcattattagatttttttcagtttataagccacacaagaaagttaacccatgtcatctctagaggcataaaaatagacagttgttctgagaaacatatctgtttattcctagttcagtaacagcgcaatgagatcctaaattatgaggtactttttgtttatcttattgtctcattggcaaccagcaactcctgttgcatttttgacatgggaatctatcagggtgtggttaataaagtacgtttccatggtaagtattctttaagaaaaaataaaccaagatttcatcagagacagaaattactgcaaactgtacttaccagtttctggtgatttgtataactgagctttcgatttcaaaaattaaaaagttctctttagtgtaacatatttttgtgtctagtaattttacttcaaaaataaaagagaattttctttaccatcaaacaacgttgctgtaatgagtggtgatatatttagtcctagaggaccaaagagaaataaaatggctttgattatgctggtttaagaaaatatatccgattaaacagaggttaatatttgtgatgtttgatttagtaaaattataagactaccaaagaaaactagaagagcaaaataaccaaaatttatataaagatttcaatgttgataattcaaaggtttagaattttttccacctttcaaatactaagttgctgttatagatttactaccattattacccagattttccattttactaaaaaataataacacagttcgttatgataaatcagtatagatctttaggctcaacacaaccctgctaccttttaaacttttttattatacaacaaacacatatgcattatcaagaaaagatgagtttagcaaaatgaagaaaaatagtcttatcaactagaaataagcaccattaaaatttctgtgtgttaaaagataatatgctgctgaaactttttttttcttgaaatgtccctcttttctttcacttttcattttcttaatccctaaccttctactgttgaattttctggaccaccgttataccccctcatcaatagggcatttttttctgcagtagctgagcacaatggtcatattgaagaaattcccttgtgggggctttgcctagaggtaaaatcctcttggccgggtgcgttggctcatgcctgtaatcccagcactttgggaagctgaggcgggagcatcttctgaggtcaggagtttgagactagcctggccaacatggtgaaatgccgtctctactaaaaatacaaaaaattagccatcatagtggtgcattcctgtaatcccacctactcgggaggctgaggcaggagaattgcttgaatgccggaggcagaggttgtagtgagccaagatctagattgtgccactgtgctccagcctgggtgacagagcaagactccgtctaaaacaaaacaaaacaaaacaacaacaacaaaaacaaagtaaaaccctcttcactgtgatgactctaaagcttgcttttgtctctctaaatcacaacaaaccgtttgcagtgtgatgctgtgctcaaaattgagaattctcactgactgttccaggcatctgtctactcattcttgccatgctgagggctgtcatttctcaccactgaatgcctttttttttttttttgtgcttctttttaaaagtcttttcctgtttgctttgaacttcaataaaaacagggggtaagaaggtaatatgcttttattttgtctgtggggtttttttcttccttcttttggttccttttcagatcctagctcatcaaggccatttgccacactgacactggaataagcctgaatgggggtctatatttcttccagtagttagtttaattcggtcattgagctgttgctctatttgtaaccgtcatgctagatccgagggctgactattcctgagaaagtgtggagatcctttcagactgccatggtgctagggcagagtttcagtcattatcttctgtgtctgctaattcttgcatcatttcttctactctgtaacctggttggctatgaaaagtgattcttcctcactgatgctgcaggcaatgggtgaatatccaattgttctctcaggttcttgtcatcggatacaaagaacaaatttcatgtgtccctgtttctgagaggttcaaagggagcaaatgcacagattctagcttaggatgacttccagcttctaactatttgtctgtgcattccatttcctcacccctgcgagaggttgcagtcatggtaacggctgttctgagaaattcagtgcccaaactggagggaggggaacacaaactcagcttcttcccaagcttcagggcagggctggcaagttttgccttccccgccagcaagagccctggctttaggggtcttgctacagttttcccagactggaggtcttggggctggattctggttcttagcgaattcttgccaaacacctcccctttccccagtatccatttgttttgaaggttctttgcaggcttcttgccaataattgtcttaaaacaaatggagttgtttgcatacctagatggagtaaaatatttaggaaactattctacaaatatgaaacagcccagatttgggccagaagtcctggtcctgagtctcttgttagccactcttaactgtgaattattggacaaatcatttctcgcactctgtgtaatctgtcacctgggtacttgatgatttaaaccaggaattgtggcccttctcataatactgtctcctaaatattgggtgaatttagtatatcaaggagggcagggcactatacccgtaggggactctctctctccctctgtctctttgcctttctcagatatgcatctttggtggcagacctggggttcaaaccacagttgttttgaaatttctctttccttctccactatgctccccctcactgaaactttaataataaaagtagcacacttattgggcatgtatttgttatgttccaggttctgtgctaacagctttaagtgcattattccacgtattttcacaacaagcctgtgaggaagaggttatttccacttatagataaagaaactgagacttggagaatgtaagtgattttacccacaaacacagggcagtaagtgggaggccctgttcaccttagaacacctggcttctatcattcatggtgctgtttctagtgtggtttgaagaacctgagatccaaactgctatacaagtgacctgctgctttcttggagagagaaatctgaaagacagtggagccctcacattgtctttgcctgtgcacagcagatactgccaattttgggtgggttacacttgtttaccacgttgcttacttcagaaatcaataaaattctcttcatgcttttattttacaggttcttacgtctgttgctagattatcgtccaaaagtgttaatgcccaagtgactgacatcaactccaagggattggaattgaggaagactgttactacagttgagactcagaacttggaaggcctgcatcatgatggccaattctgccataagccctgtcctccaggtatgttacacaaaacatccagagattacagtgaaagtcacagttaggagtagcacatagtaatcatgactataataattttacagtttttggttcccctatattatataacataactgagagaaaaacaactatgaaattattttccaaagatgagttttatttatatttatcacgcttatttgatgtggttatggataaatttaatttacaagtgacatgcacctctgaaatgagaagactggtctatttggctccatttttttctaagcaaaaatgactcatttgtgaatatgaaagcttatcatgtcaaaactgattaggatctaccataacaatagtggaactctgaaagcaagagacttctcctcttaagggatatgtctcagcacttcccttcaatcagaaggtctctaagaaagctgtcctggcactgaagcctgcaaaatatatttgcattttgcagctgaataactagacgaataagaaaagcaacaggcagaaaccaacagcttttttttttaaatcaatactttgaagggcaaactaatttcagatcacaaatccttcaaacactggtaaattagaagctaagcaaaatgcaattgtgcaaacatttcttcaactttgagtagagttgaaaaaaggaaaatagcacatgggttcatgtgatagattaatagaacaaaaaaggactgtagtatgtttcatggttgtcctcccaggcttccagcactcaagctgtaaaacaggatgggtcctgtttgtgaaaagattctaccagtctccctgtgcttcatgtccagattttaataaccgttatggaaaaatctcccctataacccaacccatttatacttaaacccatttacacttaaatcttctccaacacgaataaagaatggcgtcctggctttattataacctgctaaacacaatcaatttgctctatgaattgaaatctttgttcataattttaaaacctaaaatgtaaattaaaatgaacaaaatatgctttgcacatcaaactgtcaaggatttttgtctattgctttttcggttttgctttgttttattttaatttaagaagtgaatgtaaattgttacaaacttttaagaaatacatttggatatatgagtcaaaaatttgaaaaaaaattttttttaacttttttttcttattattatactttaagttttagggtacacatgcacaatctgcaggttagttacatatgtatacatgtgccatgctggtgcactgcacccactaactcgtcatctagcattaggtatatctcccaatgctatccctcccccctccccccaacccactgtttatacttgctaagatagcaattaatttatacgaaaataccctgagaaaataatcaaaaataaaggaaaatattgtacacatgatatttcattatcaatatgatgtaggattccttaggtaaattctggaatatttaaagactgaaatagtatgtatcaaagtcatgttttctgatatatttctaatataatcctcaattatataaaccttcttaagtaatttccaactgatctggctggattaactgattctccctgatccgtctataaaatatcctggatggcatccaaccatgttaaatactcttcccacttcaattccctcttgttgaattttaggcttatcaatatctaagttgcttccaaatctgtttatgctcattagtgttgtgatattcatatgtaatttgattgaatattgcataaattaatgaaatacaaatacagagaaaagacagattttatttctaagaaaactaggttgagtgctttggagaattcaataaagatgagctgctaaaatgttgttgatgaattttgtgagggaaaaataaaagttaagtagtaggaaaaatcacaaaaatctctaaagccattcatacagattattggtatgtttatgttttcctttcatcaaaaacgaagggagggagggagggagggagggaggagaaaaaaagaaagaaatgaaaaaggaagagaaaaatagacaaacaggaaattataggtgatgcattatggatgtgacttaagaaagaacctgggattataataacttctatatcacaaaattgtcaaatgattatatagttatattttataaattaaaacaaaatatttacagtatttttaagtgatttcctatgtaaattgacatttttgattaaatgatcaaagaagtcctccccacatcatcatattcaataagaaggattctatggtatactgcacacatatttcatatatacacattgtacatatatatatatacacacctgttatatatatgctcatgcatatacatgtatgtatgtattatatgcataggaatgcaacaaaataacggcaatatctccagattgttgatatatagttttttgtttgtttgtttgtctgtttttttgagatggagtctcactctgtagtccaggctgaagtgcagtggcatgatctcagctcactgcaacctctgccttctgggttcaagcaattctgcagcctcagcctcccaagtagctgggactacaggagcacaccacaacaccgggataattttttgtgtttttaatagaggcggggtttcaccatgttggtcagtctggtctcaaactcctgacctcaggtgatctgcctgccttggcctcccaatctgctgggattacagacatgacccactgtgcccggcctgatacataagttcttattatactttcctgccttttccaacatttctataataagcatatgtaccattatactaaaaaaagtattttaaaatctgattttcaataaaacaactacttattttcttattttaataaattttgttttcatactttggaggaacgaaagaacctgtggtttactgtatctccattataccaaggaagttgtgctaggcacattctgtaagataattcattaaagagaccaattcattcagagaccagtataataccactagcaaaaaataaaattatacttggacttgggttctgttttctttaaatattgctacaatgttatatagccctatgcactatttttgaattttgttctgggaatctccagtttgttttttactattgacagactattatctttacttactaaaatagcatatatatttacatgtcataatttgctcatgtagtaaatattttgttttatcaaaaagaatatatgaagttattaaggaattcatgttcctaggaaacacagcagaaattaattttgctgtataggtgagcagagcagattaaactgcttatatttttaaaactagttataccattaacaattagagtcactagtaatgtttttctcacacttcccttgatattttctgtcactttcttcttaattctctattcccttaccttcatctagctttccccatagcatttttttagcacttgatatcaatatatttatttgtgtattgtctgcctgcatcccaagaaggtgaagtctttggtgatgaagggtttatcttatttcctgcataatatcaaacttctaaaaagttcctaggactcaataggcatttaataaacacctatcagataaatgaattttttaagagaacacattacataatgctttctaatagcaatacagtgacaaactttatttttaaagtaaattaatgcaaatatgtatattatttaaaattgtttgtttttaagataacaagcctgatagataataaattttggcataaatatgttaatgcttatcaaaactcatcagtacatatcctgtcttttgatgaaaaagtaatagaaaggggagaacaaagagaactctcatgctcactaggcaggccgagtgcagctctgacagcaatgcaagagaaatattactatctccatcatatgcaggatgaaatttaggcttggagaaaacaacactttgttaaagattttatagctgctcaagatttgaccaaggcttttaaaacttcaaatgttcttttccttattacactgtttcttatcaggatttgtggactttggaaagtaaaatgaaaagtcagagcaatgtgagttagtttgataatctcagtggccagtaacagctatccccactctgaatatataccaaccaacctctcccctttctccaaggaagttagttcttggcaaaggcatttcatgcagtgtactttgtcattctaaccttcctttttttttgtatgctcctgttgctaatcatcctatttctatgggcttcagtcactcttttagcttttgtcttgggagactttctgtctgttgacatgacttttaaaataatcttctcccccattgtatttatatctcattagcctaccccccctccccttgtgttttagaagagttttattgtctgtcatccctctatacttcccaccctgttacctgcccgtgtcctgttcaaacacttgctccttttttccttgggcaggtgaaaggaaagctagggactgcacagtcaatggggatgaaccagactgcgtgccctgccaagaagggaaggagtacacagacaaagcccatttttcttccaaatgcagaagatgtagattgtgtgatgaaggacatggtaagagtcttaaaatgcaattgaaagaggccaatcttggaatttcatgtagaaccatttataagacaatttgaaattggggcctactgtggtgctatgttgacacacaggaaagggaaggacaggtggctagggtaccgcagaaccaggtgccgagctaactactggtctagacctttatgagtaagtctaggcaattcttccagatataggagaatgagtaaatatgaaccctaggaacagggttcatcagctcaaatcaaaagctcagaaattattttttttctggccttgacttacgcttatataatggtgctcgttcatggccagaaaaattcagaagcctgcagatacaaggacacagaaatccaaaaggtaggtaggaatgctccccctttctttggggatttagtttgtcctgataattccatctggggacctgaattttcatggatatctcaatgtattctaaggacccagattgaagtataacagaagtgtttctagttttgtttgacatgaagaaacctctggacataaagcttttcccatccctattcagcctataaagcaatgtttctcatccctggcagtgcattagaattgcttagggagctactaaaaatgtttcatgtttaggtcccatctcagggcaattacatcagagtctctgagtgaggaccttggtattggcctgatggagtaagtctggggtagagcctaacattctacatttttaacaagctcccaggtgatgctcatgctgccagtccccaggtcaccctttgagtgttaaggctagagactagaggaacaggggagacgtctaagtattggtgaggtttgtcttggttgctgctaaatgattgctggccatttcaacctggggtttcctgttctgtgtttaaacactgactgtattactggtgtcatgctgtgactgttgatataagcagtggatctcaaaaatccatgcagctcctgcccaccattttcatagtctgcttataattagccgctataactaatagtttccaaactgattttctaggcttagaagtggaaataaactgcacccggacccagaataccaagtgcagatgtaaaccaaactttttttgtaactctactgtatgtgaacactgtgacccttgcaccaagtaagttttagtctttctctgattaaaacactagatataacatgagagttatcattttcctagggaagtaacactgactgagagttaagaattagggttctagtcctgctttgcctccaagcaactagatgactgtttgctcatttaaacactggtgaacttcctgtgtaggtgacactttgctggccacttaagctaaagatatgagccagacatatgcctggcatcaaggtatcagtccagaagcacagacagtcaaggaaagcaatggttattgtgtttcaacagcatgagaatagcatcagaagagacttatgttccaggtgttacaaattatacagaaaaacttttcaactctgcctgaggagatggaggtgaagacatgaatgtgtctcatgacttgaatcttgaagaaagaaaagaaagagcattaacaggtgcacatggatggaatgcacattataggcagggaaacagcagctgcaaagagacaaaggaagctcaggaaaacagaatacctggctggagtgtaggaaagactgagaagcgttcagggctgaatcatgaagggccttgtcttttaggctaaagaggttggttttgacccaaagttagagaacctgccttgtaagtcatcttgggaagcttttaggagttcatctaataggcttaagaaataagcacatgatgccttatgttttatttgctaggaaaattttatgtacagttgtcagttgtcccaggctctcccctggccacaagtgtctgagagtgcccctatgatgacaggctaggtaggcattggggaagcagctgacttagtaaaataataacaataatagtaattccaaatatttcataatcttgatttaattttcttgtcatcataaccttttgaaatagataatattatttccatgttattgatgagataaaaggctcatggcagtgtgacttcccaagctcacctagtcaggtaatgggcagaggctgtgaactcagacaacctgattgtgaatgtttgtctgtctgaaggaaatcacacatgaacctcttgagtctcctgatcaccaccggttgctaaaagtggcagcctctctaagggccagctgagtaccctccctgagctacatcatgggcgtggctatcacctggccattttcttggtctataggaattttttgaaattacttttgacagtttattttaagagctagtttaagctataggatttacgtgttcagtttattactaggtttaagtttatttttgtatccacttcatctctcttgtgtgtcactattttcctatcttcctttaactcttgaaatcttaagacagagtcattccttatgatatttttcatccagccatccaaattatattaacttgtgccagctttagatactaatttagaaatatttgaaggaatacgtttgccagagatgcaaagatgaataaaatggcccctaatttacaaagtgccattgaaaattataaaggaattattctgccaggcttttgaatttctcctgtatttttttttctagatgtgaacatggaatcatcaaggaatgcacactcaccagcaacaccaagtgcaaagaggaaggtaattatttttttacggttatattctcctttcccccaaccccatggaaagatgtgaagaaaaaccaatcactcttgattactagaaagtcctttatttaatcttaaagattgcttattttcatataaaatgtccaatgttccaacctacaggatccagatctaacttggggtggctttgtcttcttcttttgccaattccactaattgtttggggtaagttcttgctttgttcaaactgcagattgaaataacttgggaagtagttcacaaagatttgcctcattcttacctataaaaagctaccactttggtagatttatgtattgttaatttcttgcccctgaatgcagccttgagagctgactgataagaacaaatgaaattattcctcagctagtttctgagcaacagttttggggcattgagtggtattctcatccttcctatgaacaggtgttctctgcagcagcagaattggccaaaaatcagaagtaattcttcactattcattgagatctccctatgcaaaaagagaacacaagaagcaaaggcattcccaggaaacacattgcagggaacactttaaaaacttgtacttcactgcctcctcttcctcggcctaattgcttgtttttaattatttctccttcttaacttaaaatactatggggacacatgttatacaaaggtgacttagtagagtcagtagaaaagccaaaattagatattatcataattagtctagaaaaatccctttaagtcattcatcaactacagggtcacaccaactttcagtaacttagaagtattcaattttcccttctcagaacaattatctgtttcttcagttcagttgaagaagaaagtttgccttgcctttagcggttgtttagctgaaaatacatttgggatatttaagcactgtaattgtgctcagagacatacagattcttctatctcacattgactttaatgcatacacctattgagtatgtatgcttgagttatttgtgtgtgtatttcatttctgggcatccatagcaagttgatgttgacttgcttgtcctacggcttctgcatcctgccatagtcttgccgtccacatctttgctggacagagagtggtgcttgccatatggtaagtcaaaagccatctccttgctaggccagcctgtggtaattagatgactaattaagatatgtcctttcactagaacacttgacttagtagtacgaaagttccaaaatcagcggtctcctgcgatgtttggccacttttaagtttcactgaatttctcctttttccttcttatatttctcttagtgtgaaagtatgttctcacatgcattctacaaggctgagacctgagttgataaaatttctttgttctttcagtgaagagaaaggaagtacagaaaacatgcagaaagcacagaaaggaaaaccaaggttctcatgaatctccaaccttaaatcctgtaggtattgaaataggtatcagctttccttgaaaagaaaaatagagaaattagtgatttggctttttgttacttccttttacttttttgtttcttgtttgtttcattttgtttgagatggagtcttgctccatagcccaggctggagtgcaggggtgcaatcatggctcactgcagcctcaaagtcctgggctcaagtgatcctcctgcctcaactgtgaccctgggactacaggcatgcaccaccatgccaggttaattttttatttttattttttatagagacagggttcactttgttgcccaggctggtctcaaacttttgacctcaagcaatccttctgcctcagcctcccaaagttctgggattataggtgtgagccatcacatccggcctgttactttctttatttaaacattgcttgtgtttgtgggtgcataggttaaaggggcctcacagatgaattatgatatggtacccaaattaaaagtaaacttgtacacaaaaaccgtaagaatcaacaaaggagggaattgagagtgtgctgaaaatgagctgaaaaaagaacttggccaacctccatgttccgtgttgttgttatccctgcagagaaacagcctttaccaggcatgggttcagtatccaagcctgagcaaagagattgctcttaaagtttgtcataagggcacagcttaactttctctggctgccggttgaccttatgtctcaccatatcttttcccaacatggtatcaatatatttcagatagcaagaaatacaccgagaaaagaaaccagttgcacttaatggaattattttcttatttggggccagggctcagtgggagttaggattctttaaattaatatggtaaaaaaaatttcaatttgagccattttctggtattaagaataagaatagttacagggaagaaaattttagagtagttatatttattatttattttaataagtctatatgaaaaataacctcaacctattttatgaattctttaggtttcttgcctttaaaaactaagacaatattgcttagtttctggcaaggccggaacctttcagaataaaaattgaatggtaaaagtaaccttcttaatcacttaatctagcttcctaattttatacatcaagcaactgattgtacttctttctgaattaaggaaaaattagaagttcacatttagaatattctaaagatatatttttatttgtctttctctgcttccattttttgctttctaggaaacagtggcaataaatttatctggtaaggcttttatcattttatttcatagagatggcatcctttagagtaataggccaatttcagagtaaaataatgttactaatttcagtgacatattatgggatcttgttatttctcatacaattctacctgctcagcataaagcatttatcaggcagtttgtttaaatttataatgagtactcatagttaaaaataatcaagtaacaataagacacaatagtctgaggcttaagaaacttttccttcataatcagctagatgtattacagaactcctgcctaaaaagatctagaggttaaagtgtactgtagactcaggtattatcagtgtacccaactctataacaacatacatgattccattcagttgttctttgatctgtgatttagagataagatgatcataaactctttgcttatacttttagatttgtgggtcattgatcattggatccataagagatgttataggtgatcttttccaatcctcttaatttataaatgagaaaagaagatacagaaatgttatgtattgtggcccaggttatttaacaaattactgggtatatggcaggatttggagttagaactcaaggttgtcttgtttctgtattcccctagtcagctcttcatagacctttaggacttagctatattctgaagtactataaaaagagaaataaacatggttttcactaatgggaatttcatttagaaaaacaaattttcagactattttctatttttcagatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccaaagcttggtctagagtgaaaaacaacaaattcagttctgagtatatgcaattagtgtttgaaaagattcttaatagctggctgtaaatactgcttggttttttactgggtacattttatcatttattagcgctgaagagccaacatatttgtagatttttaatatctcatgattctgcctccaaggatgtttaaaatctagttgggaaaacaaacttcatcaagagtaaatgcagtggcatgctaagtacccaaataggagtgtatgcagaggatgaaagattaagattatgctctggcatctaacatatgattctgtagtatgaatgtaatcagtgtatgttagtacaaatgtctatccacaggctaaccccactctatgaatcaatagaagaagctatgaccttttgctgaaatatcagttactgaacaggcaggccactttgcctctaaattacctctgataattctagagattttaccatatttctaaactttgtttataactctgagaagatcatatttatgtaaagtatatgtatttgagtgcagaatttaaataaggctctacctcaaagacctttgcacagtttattggtgtcatattatacaatatttcaattgtgaattcacatagaaaacattaaattataatgtttgactattatatatgtgtatgcattttactggctcaaaactacctacttctttctcaggcatcaaaagcattttgagcaggagagtattactagagctttgccacctctccatttttgccttggtgctcatcttaatggcctaatgcacccccaaacatggaaatatcaccaaaaaatacttaatagtccaccaaaaggcaagactgcccttagaaattctagcctggtttggagatactaactgctctcagagaaagtagctttgtgacatgtcatgaacccatgtttgcaatcaaagatgataaaatagattcttatttttcccccacccccgaaaatgttcaataatgtcccatgtaaaacctgctacaaatggcagcttatacatagcaatggtaaaatcatcatctggatttaggaattgctcttgtcatacccccaagtttctaagatttaagattctccttactactatcctacgtttaaatatctttgaaagtttgtattaaatgtgaattttaagaaataatatttatatttctgtaaatgtaaactgtgaagatagttataaactgaagcagatacctggaaccacctaaagaacttccatttatggaggatttttttgccccttgtgtttggaattataaaatataggtaaaagtacgtaattaaataatgtttttggtatttctggttttctcttttttggtaggggcttgctttttggttttgtcttccttttctctaactgatgctaaatataacttgtctttgatgcttcttggatcccttagaaggtacttcctttttaaccttaacccttttagtagttaaataattatttccataggttgctattgccaagaagacctcttccaaacagcacatgattattcgtcaaacagtttcgtattccagatactggaatgtggataagaaagtatacatttcaaggggtaggttttattattaagaaagccaaatgaggattttgaaatattctttcctgcatattatccattctagctacatgctggccagtgggccacctttcttttctgcaatttaatgctagtaatatattctatttaacccatgagtcccaaagtattagcatttcaacatgtaagcatgtcggtaagatagttgtgctttgcttagggttccctcctgtgttatggtctggaaagtgtctttaggcagaaagtctgagtgatcacagggttcgctcattaatttctcttttctgagccatcatagtctgtgctgtctgctctccagttttctatttctagacagaagtagggcaagttaggtactagttattcttcatggccagaagtgcaagttctactttgcaagacaagattaagttagagaacaccctattccactttggtgaactcagagcaagaactttgagttcctttgggaggaagacagtggagaagtctttgtacttggtgatgtggtttttttcctcatggcttcacctagtggccccaagcatgacttctcccatgtcaatgagcacagccacgttcccgagttgaggtgaccccacggtccagaatcatcctcattctggtgaacctggttctctttgtggtgggcatactgggtaggagaatcacccaaaggtcacccatgagctgcagaaaaaaaggctatttgcagaaggagctcacagatcacattgaaagcattgcatattcaaacatcttggtcttctttattggcatgcccacagggtcttctgacctctgattagatcagacactttttagatattgaatcatcagtttctgtacaactatctgaataaggtatataatcaatgaaatttagaatttttttctatgcttactcctgattggtaatttgtttgggtttagaattctatacaaggccatttgtaattttcctcagcactttaaaaatattaaaccatgttttcttaacatctgttgctgttgattatgtctgctcttggtgtaaatatcattcctttgtaggtaatctgtaattttttttcctctggtaatttttaaagcttcccctactactttcctatgtgttctgcagtttcacaacaatgactttcaatgtgtatttatttttacttaatctatttaataagatttaattagaaaggcagggctttcatcatttctggaaaaaatcccagctattttctctttaaatagtgtttctttaccattctcattctcttcttctgaaactgttgttattatctgttgaagcttcttgagttattttctaggtctcttaattttttttaaaacaataattgtctccgtatgttgcattttaggaaatttctcattactctttttaaattaactgttagtttttactaagcccggctagaatagtctatccactgaggtatgtattttgttttctatatttttcattcttaatgtttcttattgttccttttacatttttacttgtatatgtttcattcctgctttttttaattttctttcttctttctcttttctctctttttttttttcttcttgcagtgcagacatcgactttattagtacagcagaggtgcccacagagcaggcagaagggatcagtgcctggatctgtcccttcctccttctgccctcgctgagccctagtgtgtccggaattggtgggttcttggtc

SEQ ID NO: 20 - DNA, 2534 bp, Homo sapiens
  misc_feature (1)...(2534): Fas antigen
gacgcttctggggagtgagggaagcggtttacgagtgacttggctggagcctcaggggcgggcactggcacggaacacaccctgaggccagccctggctgcccaggcggagctgcctcttctcccgcgggttggtggacccgctcagtacggagttggggaagctctttcacttcggaggattgctcaacaaccatgctgggcatctggaccctcctacctctggttcttacgtctgttgctagattatcgtccaaaagtgttaatgcccaagtgactgacatcaactccaagggattggaattgaggaagactgttactacagttgagactcagaacttggaaggcctgcatcatgatggccaattctgccataagccctgtcctccaggtgaaaggaaagctagggactgcacagtcaatggggatgaaccagactgcgtgccctgccaagaagggaaggagtacacagacaaagcccatttttcttccaaatgcagaagatgtagattgtgtgatgaaggacatggcttagaagtggaaataaactgcacccggacccagaataccaagtgcagatgtaaaccaaactttttttgtaactctactgtatgtgaacactgtgacccttgcaccaaatgtgaacatggaatcatcaaggaatgcacactcaccagcaacaccaagtgcaaagaggaaggatccagatctaacttggggtggctttgtcttcttcttttgccaattccactaattgtttgggtgaagagaaaggaagtacagaaaacatgcagaaagcacagaaaggaaaaccaaggttctcatgaatctccaaccttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccaaagcttggtctagagtgaaaaacaacaaattcagttctgagtatatgcaattagtgtttgaaaagattcttaatagctggctgtaaatactgcttggttttttactgggtacattttatcatttattagcgctgaagagccaacatatttgtagatttttaatatctcatgattctgcctccaaggatgtttaaaatctagttgggaaaacaaacttcatcaagagtaaatgcagtggcatgctaagtacccaaataggagtgtatgcagaggatgaaagattaagattatgctctggcatctaacatatgattctgtagtatgaatgtaatcagtgtatgttagtacaaatgtctatccacaggctaaccccactctatgaatcaatagaagaagctatgaccttttgctgaaatatcagttactgaacaggcaggccactttgcctctaaattacctctgataattctagagattttaccatatttctaaactttgtttataactctgagaagatcatatttatgtaaagtatatgtatttgagtgcagaatttaaataaggctctacctcaaagacctttgcacagtttattggtgtcatattatacaatatttcaattgtgaattcacatagaaaacattaaattataatgtttgactattatatatgtgtatgcattttactggctcaaaactacctacttctttctcaggcatcaaaagcattttgagcaggagagtattactagagctttgccacctctccatttttgccttggtgctcatcttaatggcctaatgcacccccaaacatggaaatatcaccaaaaaatacttaatagtccaccaaaaggcaagactgcccttagaaattctagcctggtttggagatactaactgctctcagagaaagtagctttgtgacatgtcatgaacccatgtttgcaatcaaagatgataaaatagattcttatttttcccccacccccgaaaatgttcaataatgtcccatgtaaaacctgctacaaatggcagcttatacatagcaatggtaaaatcatcatctggatttaggaattgctcttgtcataccctcaagtttctaagatttaagattctccttactactatcctacgtttaaatatctttgaaagtttgtattaaatgtgaattttaagaaataatatttatatttctgtaaatgtaaactgtgaagatagttataaactgaagcagatacctggaaccacctaaagaacttccatttatggaggatttttttgccccttgtgtttggaattataaaatataggtaaaagtacgtaattaaataatgtttttg

(19) United States
(12) Patent Application Publication
(10) Pub. No.: US 2008/0090234 A1
(43) Pub. Date: Apr. 17, 2008

(54) Diagnostic assay for autoimmune lymphoproliferative syndrome (ALPS) and genetically related disorders

(76) Inventors: Kejian Zhang, Cincinnati, OH (US); Richard J. Wenstrup, Cincinnati, OH (US); Jacob J. Bleesing, Cincinnati, OH (US); Alexandra H. Filipovich, Covington, KY (US)

Correspondence Address: TAFT, STETTINIUS & HOLLISTER LLP, SUITE 1800, 425 WALNUT STREET, CINCINNATI, OH, 45202-3957, US

(21) Appl. No.: 11/580,529

(22) Filed: Oct. 13, 2006

(51) Int. Cl. C12Q 1/68; C07H 21/04; C12P 19/34

(52) U.S. Cl. 435/6; 435/91.2; 536/23.1

(57) ABSTRACT

The methods and compositions of the invention find use in the clinical diagnosis of TNFRSF6-related syndromes, particularly autoimmune lymphoproliferative syndrome (ALPS). The compositions of the invention include isolated nucleic acid molecules and oligonucleotide pairs suitable for use in amplifying regions of the TNFRSF6 gene and in determining the nucleotide sequence of the TNFRSF6 gene in a patient. The invention facilitates efficient, cost-effective amplification of one or more regions of the TNFRSF6 gene. The nucleotide sequence of amplified DNA comprising one or more regions of the TNFRSF6 gene can be determined. Knowledge of the patient's nucleotide sequence in the TNFRSF6 gene allows diagnosis of the patient.

### FIELD OF THE INVENTION

The field of this invention relates to the field of genetic diagnostic assays for primary immunodeficiencies.

### BACKGROUND OF THE INVENTION

The physiological death of cells in a living organism in the natural course of events is known as apoptosis, and is distinguished from the pathological death of cells, i.e. necrosis [Kerr et al., (1972), Br. J. Cancer, 26:239]. Apoptosis is an example of programmed cell death, which is where certain cells are programmed, in advance, to die in a living organism in the natural course of events, such as when the cell in question has performed a pre-determined function. Apoptosis is characterized by such morphological changes as curved cell surface, condensed nuclear chromatin and fragmented chromosomal DNA, amongst others.

Apoptosis plays a role in the differentiation of lymphocytes (T cells and B cells) by eliminating cells that recognize an autoantigen. In this respect, it has been demonstrated that 95%, or even more, cells, such as those which react with autoantigens, are eliminated in the thymus during the maturation of T lymphocytes [Shigekazu Nagata, Tanpakushitsu Kakusan Koso, (1993), 38: 2208-2218]. When such cells are not eliminated by apoptosis, then mature, auto-reactive lymphocytes remain in the system [Nakayama et al., (1995), Mebio, 12 (10):79-86].

Various molecules have been identified as being involved in apoptosis, including: Fas [Yonehara, S., et al., (1989), J. Exp. Med., 169, 1747-1756]; tumor necrosis factor receptor [Loetscher, H., et al., (1990), Cell, 61, 351-359]; CD40 [Tsubata, T., et al., (1993), Nature, 364, 645-648]; and perforin/granzyme A [Jenne, D. E., et al., (1988), Immunol. Rev. 103, 53-71].

Fas is a transmembrane protein, present on the cellular surface, and binding of its extracellular domain to a protein generally known as “Fas ligand”, expressed on the surface of other cells, induces apoptosis in the cell expressing Fas. Abnormalities in the Fas/Fas ligand system result in various disorders, by failing to delete cells which could be detrimental to homeostasis, and which should have been eliminated by apoptosis, or, alternatively, by inducing apoptosis in cells not otherwise scheduled for elimination and which could be essential for maintaining homeostasis. Such disorders are those referred to herein as being conditions arising from abnormalities in the Fas/Fas ligand system.

In the development, or progression, of diseases arising from abnormalities of the Fas/Fas ligand, it is often the case that abnormal cells, which express Fas but which, nevertheless, remain undeleted (abnormal cells), either attack normal tissues or cells, or else proliferate abnormally, thereby causing disorders in the tissues or cells which, in turn, lead to the respective disease symptoms. In some cases, these disorders may arise from, or be exacerbated by, the expression of Fas on the abnormal cells, thereby stimulating apoptosis in normal tissues or cells. Specific examples of diseases associated with abnormalities of the Fas/Fas ligand system follow. Additional conditions correlated with Fas/Fas ligand abnormalities are described in U.S. Pat. No. 6,972,323 herein incorporated by reference in its entirety.

Links between various human autoimmune diseases including, but not limited to, Hashimoto disease, systemic lupus erythematosus, Sjogren syndrome, pernicious anemia. Addison disease, insulin dependent diabetes mellitus, scleroderma, Goodpasture's syndrome, Crohn's disease, autoimmune hemolytic anemia, sterility, myasthenia gravis, multiple sclerosis, Basedow's disease, thrombopenia purpura, rheumatoid arthritis and abnormalities in the Fas/Fas ligand system have been reported many times. In the human, several cases have been reported involving swelling of the lymph nodes, hypergammaglobulinemia and marked increase in CD4⁻-CD8⁻⁻ T cells [Sneller, M C., et al., (1992), J. Clin. Invest., 90:334]. These cases were reported to be based on abnormalities in the Fas gene [Fisher, G. H., et al., (1995), Cell, 81, 935; and Rieux-Laucat, F., et al., (1995), Science, 268, 1347], and designated autoimmune lymphoproliferative syndrome (ALPS). Additional links between abnormalities in the Fas/Fas ligand system and disorders including but not limited to, insulin dependent diabetes mellitus, graft versus host disease, allergic diseases, rheumatoid arthritis, arteriosclerosis, autoimmune heart diseases, ischemic heart disease, viral heart disease, dilated cardiomyopathy and chronic cardiomyopathy have been reported. Myocarditis is inflammation of the heart muscle considered to be caused mainly by viruses, such as coxsackie virus, and is typified by chest pain, arrhythmia, heart failure or shock, after cold-like symptoms. Cardiomyopathy is defined as “a disease of the cardiac muscle of unknown cause,” although its cause is also considered likely to be as a result of viral infection. Additional links between abnormalities in the Fas/Fas ligand system and disorders including but not limited to, sclerosis of the glomeruli, chronic renal failure, progressive glomerulosclerosis, acute glomerular nephritis, purpura nephritis, lupus nephritis, hypoplastic anemia, fulminant hepatitis, hepatocyte necrosis, chronic hepatitis, fatty liver chronic persistent hepatitis C, chronic active hepatitis, the bystander disorder, acute hepatic failure, alcoholic hepatitis, viral cirrhosis, alcoholic cirrhosis, acquired immunodeficiency syndrome, rejection after organ transplantation,

Autoimmune Lymphoproliferative Syndrome (ALPS) is a rare primary immunodeficiency disorder characterized by defective lymphocyte homeostasis. Its manifestations are lymphadenopathy, (hepato)splenomegaly with or without hypersplenism, autoimmunity (autoimmune cytopenias and other autoimmune disorders), and a highly increased lifelong risk of lymphoma. It has also been referred to as Canale-Smith syndrome.

ALPS (MIM 601859) has been linked to the TNFRSF6 gene (also known as APT1, Fas, Apo1, and CD95). The TNFRSF6 gene (SEQ ID NO:19) has nine exons spanning about 28 kb on chromosome 10q24.1. Exons 1-5 encode a signal sequence and three extracellular cysteine rich domains responsible for binding Fas ligand (FasL). Exon 6 encodes the transmembrane domain of Fas, and exons 7-9 encode the intracellular portion. The Fas death domain is encoded by exon 9. The functional Fas complex is a homotrimeric receptor, which, when engaged by homotrimeric FasL transmits an apoptotic signal. See Jackson et al. (1999) Am. J. Hum. Genet. 64:1002-1014, herein incorporated by reference in its entirety.

The connection between TNFRSF6 and various autoimmune syndromes has led to numerous investigations of the TNFRSF6 gene sequence by various groups. The currently available means of amplifying the TNFRSF6 gene and detecting mutations in it require multiple different reaction conditions or yield limited information about the TNFRSF6 gene. For instance in Bettinardi et al only portions of exons 4 and 9 are amplified from genomic DNA (Bettinardi et al. (1997) Blood 89:902-902, herein incorporated by reference in its entirety). In Clementi et al the 5′ UTR and the 9 exons are amplified from genomic DNA, but the amplification reactions require 3 different annealing temperatures thus requiring either 3 different PCR programs either in separate machines or at separate times, (Clementi et al. (2005) Blood 105:4424-4428, herein incorporated by reference in its entirety). As the number of different PCR programs increases, the amount of time or equipment involved in determining a subject's TNFRSF6 genotype increases significantly thus increasing the cost of determining a patient's TNFRSF6 genotype and potentially delaying diagnosis.

The diagnosis of ALPS is based on a constellation of clinical findings, laboratory abnormalities and identification of genetic mutations in genes relevant for the Fas pathway of apoptosis. This pathway includes TNFRSF6 (Fas), CASP10 (caspase 10) and TNFSF6 (FasL). Thus there is a clear need for a rapid, cost-effective method of determining a patient's TNFRSF6 nucleotide sequence.

### SUMMARY OF THE INVENTION

Compositions and methods for diagnosing TNFRSF6-related syndromes are provided. The inventions are based on identification of nucleotide sequences for amplifying the exons and exon-intron boundaries of the Autoimmune Lymphoproliferative syndrome (ALPS) gene, TNFRSF6. The compositions of the invention allow amplification of the TNFRSF6 protein gene exons and exon-intron boundaries under identical enzymatic amplification conditions, thus reducing labor and equipment costs. Further, use of the compositions of the invention facilitate determination of the nucleotide sequence of the amplified TNFRSF6 protein gene exons and exon-intron boundaries. The TNFRSF6 protein gene exons and exon-intron boundaries' nucleotide sequences provide diagnostic information for TNFRSF6 related syndromes such as ALPS.

Compositions of the invention include isolated nucleic acid molecules consisting of the nucleotide sequences set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, and SEQ ID NO:16, and variants thereof. Variant nucleotide sequences of the invention differ by one nucleotide alteration from the nucleotide sequences set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, and SEQ ID NO:16, or hybridize under stringent conditions to a complement of a nucleotide sequence of the invention. Compositions of the invention include isolated nucleic acid molecules consisting of a generic segment adjacent to a TNFRSF6-targeting segment. In the compositions of the invention the TNFRSF6-targeting segment is at the 3′ end of the molecule. The nucleotide sequence of the TNFRSF6-targeting segment comprise the nucleotide sequences set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, and SEQ ID NO:16, and variants thereof.

In an embodiment the generic segment of the isolated nucleic acid molecules of the invention comprises a universal sequencing primer nucleotide sequence. In an embodiment the introduction of a universal sequencing primer nucleotide sequence in the amplified products of the enzymatic amplification facilitates determining the nucleotide sequence of the amplified region. In another embodiment the generic segment is less than 51 nucleotides. Yet another embodiment of the invention provides that the nucleotide sequence of the generic segment is set forth in SEQ ID NO:17 or SEQ ID NO:18. Embodiments of the invention provide isolated nucleic acid molecules comprising a generic segment having the nucleotide sequence set forth in SEQ ID NO:17 and a TNFRSF6-targeting segment having a nucleotide sequence set forth in SEQ ID NO:1, 3, 5, 7, 9, 11, 13, or 15, or variants thereof. Embodiments of the invention also provide isolated nucleic acid molecules comprising a generic segment having the nucleotide sequence set forth in SEQ ID NO:18 and a TNFRSF6-targeting segment having a nucleotide sequence set forth in SEQ ID NO:2, 4, 6, 8, 10, 12, 14, or 16 or variants thereof.

Compositions of the invention further include oligonucleotide pairs comprising a first nucleic acid molecule and a second nucleic acid molecule. Oligonucleotide pairs of the invention allow amplification of a region of the TNFRSF6 gene. The first nucleic acid molecule of an oligonucleotide pair consists of a first generic segment adjacent to a first TNFRSF6-targeting segment located at the 3′ end of the molecule. In an embodiment the nucleotide sequence of the first generic segment is set forth in SEQ ID NO:17. Nucleotide sequences of the first TNFRSF6-targeting segment in an oligonucleotide pair are set forth in SEQ ID NO:1, SEQ ID NO:3, SEQ ID NO:5, SEQ ID NO:7, SEQ ID NO:9, SEQ ID NO:11, SEQ ID NO:13, SEQ ID NO:15, and variants thereof. The second nucleic acid molecule of an oligonucleotide pair consists of a second generic segment adjacent to a second TNFRSF6-targeting segment located at the 3′ end of the molecule. In an embodiment the nucleotide sequence of the second generic segment is set forth in SEQ ID NO:18. Nucleotide sequences of the second TNFRSF6-targeting segment in an oligonucleotide pair are set forth in SEQ ID NO:2, SEQ ID NO:4, SEQ ID NO:6, SEQ ID NO:8, SEQ ID NO:10, SEQ ID NO:12, SEQ ID NO:14, SEQ ID NO:16 and variants thereof. In an embodiment, an oligonucleotide pair of the invention comprises a first TNFRSF6-targeting segment having the nucleotide sequence set forth in SEQ ID NO:1 or a variant thereof and a second TNFRSF6-targeting segment having the nucleotide sequence set forth in SEQ ID NO:2 or a variant thereof. In an embodiment, an oligonucleotide pair of the invention comprises a first TNFRSF6-targeting segment having the nucleotide sequence set forth in SEQ ID NO:3 or a variant thereof and a second TNFRSF6-targeting segment having the nucleotide sequence set forth in SEQ ID NO:4 or a variant thereof. In an embodiment, an oligonucleotide pair of the invention comprises a first TNFRSF6-targeting segment having the nucleotide sequence set forth in SEQ ID NO:5 or a variant thereof and a second TNFRSF6-targeting segment having the nucleotide sequence set forth in SEQ ID NO:6 or a variant thereof. In an embodiment, an oligonucleotide pair of the invention comprises a first TNFRSF6-targeting segment having the nucleotide sequence set forth in SEQ ID NO:7 or a variant thereof and a second TNFRSF6-targeting segment having the nucleotide sequence set forth in SEQ ID NO:8 or a variant thereof. In an embodiment, an oligonucleotide pair of the invention comprises a first TNFRSF6-targeting segment having the nucleotide sequence set forth in SEQ ID NO:9 or a variant thereof and a second TNFRSF6-targeting segment having the nucleotide sequence set forth in SEQ ID NO:10 or a variant thereof. In an embodiment, an oligonucleotide pair of the invention comprises a first TNFRSF6-targeting segment having the nucleotide sequence set forth in SEQ ID NO:11 or a variant thereof and a second TNFRSF6-targeting segment having the nucleotide sequence set forth in SEQ ID NO:12 or a variant thereof. In an embodiment, an oligonucleotide pair of the invention comprises a first TNFRSF6-targeting segment having the nucleotide sequence set forth in SEQ ID NO:13 or a variant thereof and a second TNFRSF6-targeting segment having the nucleotide sequence set forth in SEQ ID NO:14 or a variant thereof. In an embodiment, an oligonucleotide pair of the invention comprises a first TNFRSF6-targeting segment having the nucleotide sequence set forth in SEQ ID NO:15 or a variant thereof and a second TNFRSF6-targeting segment having the nucleotide sequence set forth in SEQ ID NO:16 or a variant thereof.

Compositions of the invention further include a TNFRFSF6 oligonucleotide pair library comprising at least one oligonucleotide pair comprising a first isolated nucleic acid molecule and a second isolated nucleic acid molecule wherein said first and second nucleic acid molecules allow amplification of a region of the TNFRSF6 gene. The first nucleic acid molecule of an oligonucleotide pair consists of a generic segment adjacent to a first TNFRSF6-targeting segment at the 3′ end of the molecule. In an embodiment the nucleotide sequence of the first generic segment is set forth in SEQ ID NO:17. The nucleotide sequence of the first TNFRSF6-targeting segment is selected from the nucleotide sequences set forth in SEQ ID NO:1, SEQ ID NO:3, SEQ ID NO:5, SEQ ID NO:7, SEQ ID NO:9, SEQ ID NO:11, SEQ ID NO:13, SEQ ID NO:15, and variants thereof. The second nucleic acid molecule of an oligonucleotide pair consists of a second generic segment adjacent to a second TNFRSF6-targeting segment located at the 3′ end of the molecule. In an embodiment the nucleotide sequence of the first generic segment is set forth in SEQ ID NO:18. Nucleotide sequences of the second TNFRSF6-targeting segment in an oligonucleotide pair are set forth in SEQ ID NO:2, SEQ ID NO:4, SEQ ID NO:6, SEQ ID NO:8, SEQ ID NO:10, SEQ ID NO:12, SEQ ID NO:14, SEQ ID NO:16 and variants thereof. In an embodiment, an oligonucleotide pair of the invention comprises a first TNFRSF6-targeting segment having the nucleotide sequence set forth in SEQ ID NO:1 or a variant thereof and a second TNFRSF6-targeting segment having the nucleotide sequence set forth in SEQ ID NO:2 or a variant thereof. In an embodiment, an oligonucleotide pair of the invention comprises a first TNFRSF6-targeting segment having the nucleotide sequence set forth in SEQ ID NO:3 or a variant thereof and a second TNFRSF6-targeting segment having the nucleotide sequence set forth in SEQ ID NO:4 or a variant thereof. In an embodiment, an oligonucleotide pair of the invention comprises a first TNFRSF6-targeting segment having the nucleotide sequence set forth in SEQ ID NO:5 or a variant thereof and a second TNFRSF6-targeting segment having the nucleotide sequence set forth in SEQ ID NO:6 or a variant thereof. In an embodiment, an oligonucleotide pair of the invention comprises a first TNFRSF6-targeting segment having the nucleotide sequence set forth in SEQ ID NO:7 or a variant thereof and a second TNFRSF6-targeting segment having the nucleotide sequence set forth in SEQ ID NO:8 or a variant thereof. In an embodiment, an oligonucleotide pair of the invention comprises a first TNFRSF6-targeting segment having the nucleotide sequence set forth in SEQ ID NO:9 thereof and a second TNFRSF6-targeting segment having the nucleotide sequence set forth in SEQ ID NO:10 or a variant thereof. In an embodiment, an oligonucleotide pair of the invention comprises a first TNFRSF6-targeting segment having the nucleotide sequence set forth in SEQ ID NO:11 or a variant thereof and a second TNFRSF6-targeting segment having the nucleotide sequence set forth in SEQ ID NO:12 or a variant thereof. In an embodiment, an oligonucleotide pair of the invention comprises a first TNFRSF6-targeting segment having the nucleotide sequence set forth in SEQ ID NO:13 or a variant thereof and a second TNFRSF6-targeting segment having the nucleotide sequence set forth in SEQ ID NO:14 or a variant thereof. In an embodiment, an oligonucleotide pair of the invention comprises a first TNFRSF6-targeting segment having the nucleotide sequence set forth in SEQ ID NO:15 or a variant thereof and a second TNFRSF6-targeting segment having the nucleotide sequence set forth in SEQ ID NO:16 or a variant thereof.

In an embodiment, the invention provides a method of amplifying a region of the TNFRFSF6 gene. The method comprises the steps of obtaining a biological sample from a human subject and performing enzymatic amplification using an oligonucleotide pair selected from the TNFRFSF6 oligonucleotide pair library of the invention. In an aspect of the invention, the method provides the step of performing enzymatic amplification using a first oligonucleotide pair of the invention. In an aspect of the invention, the method provides the use of a second oligonucleotide pair of the invention in the step of performing enzymatic amplification. In an aspect of the invention, the method provides the use of a third oligonucleotide pair of the invention in the step of performing enzymatic amplification. In an aspect of the invention, the method provides the use of a fourth oligonucleotide pair of the invention in the step of performing enzymatic amplification. In an aspect of the invention, the method provides the use of a fifth oligonucleotide pair of the invention in the step of performing enzymatic amplification. In an aspect of the invention, the method provides the use of a sixth oligonucleotide pair of the invention in the step of performing enzymatic amplification. In an aspect of the invention, the method provides the use of a seventh oligonucleotide pair of the invention in the step of performing enzymatic amplification. In an aspect of the invention, the method provides the use of an eighth oligonucleotide pair of the invention in the step of performing enzymatic amplification. Enzymatic amplification using a first, second, third, fourth, fifth, sixth, seventh, and eighth oligonucleotide pair may share identical incubation conditions. In an aspect of the invention, the temperature of the annealing incubation is in the range of 54° C. to 57.9° C., particularly 57° C.

The invention provides kits for performing a method of amplifying a region of the TNFRSF6 gene comprising a TNFRSF6 oligonucleotide pair library of the invention.

In an embodiment, the invention provides a method of determining the nucleotide sequence of a region of the TNFRSF6 gene of a subject. The method comprises the steps of obtaining a biological sample from the human subject, performing enzymatic amplification of a region of the TNFRSF6 gene using an oligonucleotide pair of the invention, providing amplified DNA of a region of the TNFRSF6 gene, and determining the nucleotide sequence of the amplified DNA. In an aspect of the invention at least one universal sequencing primer is used in determining the sequence of the amplified DNA. In an aspect of the invention the nucleotide sequence of at least one generic region comprises the nucleotide sequence of a universal sequencing primer used in determining the sequence of the amplified DNA.

In an embodiment, the invention provides a method of diagnosing a TNFRSF6 related syndrome. The method comprises the steps of obtaining a biological sample from a human subject, performing enzymatic amplification of a region of the TNFRSF6 gene using an oligonucleotide pair of the invention, providing amplified DNA of a region of the TNFRSF6 gene, determining the nucleotide sequence of the amplified region or regions, and comparing the nucleotide sequence of the amplified region with a standard sequence profile. The method provides simultaneous enzymatic amplification of multiple regions of the TNFRSF6 gene. The nucleotide sequence of one or multiple amplified regions of the TNFRSF6 gene is determined. In an aspect of the invention the nucleotide sequence of the TNFRSF6 upstream regulatory region, exons and exon-intron boundaries is determined. In an aspect of the invention the TNFRSF6 related syndrome is autoimmune lymphoproliferative syndrome (ALPS).

### BRIEF DESCRIPTION OF THE DRAWINGS

FIG. 1 presents a schematic of the amplification and sequencing test design for the TNFRSF6 gene. Amplification primers are indicated by arrows and the primer name is indicated. The nucleotide sequences of the primers are set forth in the sequence listing. The size of the amplified regions of the TNFRSF6 gene is indicated in boxes to the right or left of the schematic. Regions containing exons are indicated with open boxes while other regions are indicated with a solid line.

### DETAILED DESCRIPTION OF THE INVENTION

The present invention provides methods of diagnosing a primary immunodeficiency, particularly a TNFRSF6-related syndrome such as, but not limited to, Autoimmune Lymphoproliferative Syndrome (ALPS). The invention provides methods of amplifying a region or regions of the TNFRSF6 gene, and methods of determining the nucleotide sequence of a region or regions of the TNFRSF6 gene. Compositions of the invention include isolated nucleic acid molecules and oligonucleotide pairs useful in amplifying a region or regions of the TNFRSF6 gene, a TNFRSF6 oligonucleotide pair library, as well as kits comprising isolated nucleic acid molecules of the invention. The invention relates to methods of efficiently amplifying multiple regions of the TNFRSF6 gene. The invention allows direct sequencing of the entire coding region and intron/exon boundaries of the TNFRSF6 gene.

TNFRSF6 related syndromes are primary immunodeficiencies characterized by defective function of the Fas system, autoimmunities, chronic non-malignant lymphadenopathy, splenomegaly, and high levels of circulating αβ+CD4⁻-CD8⁻ lymphocytes. TNFRSF6 related syndromes include, but are not limited to, ALPS, Canale-Smith syndrome, ALPS Ia, splenomegaly, hepatomegaly, immune cytopenias, immune thrombocytopenia, autoimmune hemolytic anemia, neutropenia, immune hepatitis, arthritis, insulin-dependent diabetes mellitus, thyroiditis, rashes, atopy, dyserythropoietic anemia, Guillian-Barre syndrome, immune mediated thrombocytopenic purpura, glomerulonephritis, primary biliary cirrhosis, urticarial rash, lymphoma, leukemia, myeloma, and other syndromes discussed above herein. See for example U.S. Pat. No. 6,972,323, herein incorporated by reference in its entirety. TNFRSF6 related syndromes have been difficult to diagnose clinically. The symptoms associated with TNFRSF6 related syndromes include, but are not limited to, defective lymphocyte homeostasis, lymphadenopathy, (hepato)splenomegaly with or without hypersplenism, autoimmunity, autoimmune cytopenias, life-long increased risk of lymphoma, defective Fas-mediated apoptosis, chronic non-malignant lymphoproliferation, elevated αβ+CD4⁻-CD8⁻ T cell levels in peripheral blood and/or in tissue specimens, expansion of the red pulp in splenectomized patients, adenopathy, reactive follicular hyperplasia, plasmocytosis, parafollicular DNT expansion, hepatomegaly, steroid responsive recurrent fevers, hypergammaglobulinemia, anemia, thyroiditis, eczema, arthritis, and asthma, among others.

Compositions of the invention include isolated nucleic acid molecules having the nucleotide sequences set forth in SEQ ID NO:1, 2, 3, 4, 5, 6, 7, 8, 9, 10, 11, 12, 13, 14, 15, and 16, or a fragment or variant thereof. Additional compositions of the invention include isolated nucleic acid molecules consisting of a generic segment adjacent to a TNFRSF6-targetting segment wherein the TNFRSF6-targetting segment is at the 3′ end of the molecule. By “TNFRSF6-targetting segment” is intended a segment of a nucleic acid molecule having a nucleotide sequence that hybridizes to the TNFRSF6 gene having the nucleotide sequence set forth in SEQ ID NO:19. TNFRSF6-targetting segments of particular interest have a nucleotide sequence set forth in SEQ ID NO:1, 2, 3, 4, 5, 6, 7, 8, 9, 10, 11, 12, 13, 14, 15, and 16, or a fragment or variant thereof. The nucleic acid molecules of the invention anneal to the TNFRSF6 gene (SEQ ID NO:19). The invention encompasses isolated or substantially purified nucleic acid compositions. An “isolated” or substantially “purified” nucleic acid molecule, or biologically active portion thereof, is substantially free of other cellular material, or culture medium when produced by recombinant techniques or substantially free of chemical precursors or other chemicals when chemically synthesized.

By fragments or variants thereof is intended isolated nucleic acid molecules or TNFRSF6-targetting segments of isolated nucleic acid molecules having a nucleotide sequence that differs by one nucleotide alteration from that set forth in SEQ ID NO:1, 2, 3, 4, 5, 6, 7, 8, 9, 10, 11, 12, 13, 14, 15, and 16, 17, 18, 19, 21, or a nucleotide sequence that hybridizes under stringent conditions to a complement of SEQ ID NO:1, 2, 3, 4, 5, 6, 7, 8, 9, 10, 11, 12, 13, 14, 15, and 16. A fragment or variant that differs by one nucleotide alteration from a nucleotide sequence set forth in SEQ ID NO:1, 2, 3, 4, 5, 6, 7, 8, 9, 10, 11, 12, 13, 14, 15, or 16 differs from that nucleotide sequence by the addition, insertion, deletion, removal, subtraction, or substitution of one nucleotide. Fragments or variants include isolated nucleic acid molecules or TNFRSF6 targetting segments having a nucleotide sequence that hybridizes under stringent conditions to a complement of SEQ ID NO:1, 2, 3, 4, 5, 6, 7, 8, 9, 10, 11, 12, 13, 14, 15, or 16.

By “generic segment” is intended a segment of an isolated nucleic acid molecule varying in length from 1 to 100 nucleotides, preferably 1-51 nucleotides, more preferably 1-30 nucleotides, yet more preferably 1-20 nucleotides. In an embodiment the a portion or all of the nucleotide sequence of the generic segment facilitates determining the sequence of amplified DNA obtained by enzymatic amplification performed with the isolated nucleic acid molecule of interest. In an aspect of the invention, the nucleotide sequence of the generic segment comprises that of a universal sequencing primer. By “universal sequencing primer” is intended a nucleic acid molecule suitable for use in determining the nucleotide sequence of a target sequence; often these sequences anneal to regions commonly found within commercially available plasmids. Universal sequencing primers are known in the art and include, but are not limited to, M13 forward (SEQ ID NO:17), M13 reverse (SEQ ID NO:18), M13f(−41), M13f(−21), M13r (−27), M13r (−48), SP6, T7 promoter, T7 terminator, T3, pBluescript KS, pBluescript SK, XPress forward, TrcHis Forward, TrcHis Reverse, RV3, RV4, GL1, GL2, pGEX5′, and pGEX3′. Any universal sequencing primer known in the art can be used in the compositions and methods of the invention. In an embodiment an isolated nucleic acid molecule of the invention consists of a generic segment and a TNFRSF6-targetting segment and the nucleotide sequence of the generic segment is a M13 universal sequencing primer set forth in SEQ ID NO:17 or SEQ ID NO:18.

By “stringent conditions” or “stringent hybridization conditions” is intended conditions under which a probe will hybridize to its target sequence to a detectably greater degree than to other sequences (e.g., at least 2-fold over background). Stringent conditions are sequence-dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing).

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1 × to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C. Duration of hybridization is generally less than about 24 hours, usually about 4 to about 12 hours.

Specificity is typically the function of post-hybridization washes, the critical factors being the ionic strength and temperature of the final wash solution. For DNA—DNA hybrids, the Tₘ can be approximated from the equation of Meinkoth and Wahl (1984) Anal. Biochem. 138:267-284: Tₘ=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired Tₘ, those of ordinary skill will understand that variations in the stringency of hybridization and/or wash solutions are inherently described. An extensive guide to the hybridization of nucleic acids is found in Tijssen (1993) Laboratory Techniques in Biochemistry and Molecular Biology—Hybridization with Nucleic Acid Probes, Part I, Chapter 2 (Elsevier, N.Y.); and Ausubel et al., eds. (1995) Current Protocols in Molecular Biology, Chapter 2 (Greene Publishing and Wiley-Interscience, New York). See Sambrook et al. (1989) Molecular Cloning: A Laboratory Manual (2d ed., Cold Spring Harbor Laboratory Press, Plainview, N.Y.).

Compositions of the invention include oligonucleotide pairs. An oligonucleotide pair of the invention consists of a first isolated nucleic acid molecule of the invention and a second isolated nucleic acid molecule of the invention with different nucleotide sequences. An oligonucleotide pair of the invention is suitable for use as a primer pair or primer set in a PCR reaction. In an embodiment, an oligonucleotide pair comprises a first nucleic acid molecule consisting of a generic segment adjacent to a TNFRSF6-targetting segment wherein the nucleotide sequence of the TNFRSF6-targetting segment is set forth in SEQ ID NO:1, 3, 5, 7, 9, 11, 13, 15, or a fragment or variant thereof and a second nucleic acid molecule consisting of a generic segment adjacent to a TNFRSF6-targetting segment wherein the nucleotide sequence of the TNFRSF6-targetting segment is set forth in SEQ ID NO:2, 4, 6, 8, 10, 12, 14, 16, or a fragment or variant thereof. In an embodiment, an oligonucleotide pair comprises a first nucleic acid molecule consisting of a generic segment adjacent to a TNFRSF6-targetting segment wherein the nucleotide sequence of the TNFRSF6-targetting segment is set forth in SEQ ID NO:1 or a fragment or variant thereof and a second nucleic acid molecule consisting of a generic segment adjacent to a TNFRSF6-targetting segment wherein the nucleotide sequence of the TNFRSF6-targetting segment is set forth in SEQ ID NO:2 or a fragment or variant thereof. In an embodiment, an oligonucleotide pair comprises a first nucleic acid molecule consisting of a generic segment adjacent to a TNFRSF6-targetting segment wherein the nucleotide sequence of the TNFRSF6-targetting segment is set forth in SEQ ID NO:3 or a fragment or variant thereof and a second nucleic acid molecule consisting of a generic segment adjacent to a TNFRSF6-targetting segment wherein the nucleotide sequence of the TNFRSF6-targetting segment is set forth in SEQ ID NO:4 or a fragment or variant thereof. In an embodiment, an oligonucleotide pair comprises a first nucleic acid molecule consisting of a generic segment adjacent to a TNFRSF6-targetting segment wherein the nucleotide sequence of the TNFRSF6-targetting segment is set forth in SEQ ID NO:5 or a fragment or variant thereof and a second nucleic acid molecule consisting of a generic segment adjacent to a TNFRSF6-targetting segment wherein the nucleotide sequence of the TNFRSF6-targetting segment is set forth in SEQ ID NO:6 or a fragment or variant thereof. In an embodiment, an oligonucleotide pair comprises a first nucleic acid molecule consisting of a generic segment adjacent to a TNFRSF6-targetting segment wherein the nucleotide sequence of the TNFRSF6-targetting segment is set forth in SEQ ID NO:7 or a fragment or variant thereof and a second nucleic acid molecule consisting of a generic segment adjacent to a TNFRSF6-targetting segment wherein the nucleotide sequence of the TNFRSF6-targetting segment is set forth in SEQ ID NO:8 or a fragment or variant thereof. In an embodiment, an oligonucleotide pair comprises a first nucleic acid molecule consisting of a generic segment adjacent to a TNFRSF6-targetting segment wherein the nucleotide sequence of the TNFRSF6-targetting segment is set forth in SEQ ID NO:9 and a second nucleic acid molecule consisting of a generic segment adjacent to a TNFRSF6-targetting segment wherein the nucleotide sequence of the TNFRSF6-targetting segment is set forth in SEQ ID NO:10 or a fragment or variant thereof. In an embodiment, an oligonucleotide pair comprises a first nucleic acid molecule consisting of a generic segment adjacent to a TNFRSF6-targetting segment wherein the nucleotide sequence of the TNFRSF6-targetting segment is set forth in SEQ ID NO:11 or a fragment or variant thereof and a second nucleic acid molecule consisting of a generic segment adjacent to a TNFRSF6-targetting segment wherein the nucleotide sequence of the TNFRSF6-targetting segment is set forth in SEQ ID NO:12 or a fragment or variant thereof. In an embodiment, an oligonucleotide pair comprises a first nucleic acid molecule consisting of a generic segment adjacent to a TNFRSF6-targetting segment wherein the nucleotide sequence of the TNFRSF6-targetting segment is set forth in SEQ ID NO:13 or a fragment or variant thereof and a second nucleic acid molecule consisting of a generic segment adjacent to a TNFRSF6-targetting segment wherein the nucleotide sequence of the TNFRSF6-targetting segment is set forth in SEQ ID NO:14 or a fragment or variant thereof. In an embodiment, an oligonucleotide pair comprises a first nucleic acid molecule consisting of a generic segment adjacent to a TNFRSF6-targetting segment wherein the nucleotide sequence of the TNFRSF6-targetting segment is set forth in SEQ ID NO:15 or a fragment or variant thereof and a second nucleic acid molecule consisting of a generic segment adjacent to a TNFRSF6-targetting segment wherein the nucleotide sequence of the TNFRSF6-targetting segment is set forth in SEQ ID NO:16 or a fragment or variant thereof. Additional oligonucleotide pairs comprising the isolated nucleic acid molecules of the invention are encompassed by the invention. The first and second nucleic acid molecules of an oligonucleotide pair anneal to opposite strands of the TNFRSF6 gene such that they allow amplification of a region of the TNFRSF6 gene bracketed by the primer pair.

In an embodiment of the invention an oligonucleotide pair of the invention comprises a first isolated nucleic acid molecule consisting of a generic segment adjacent to a TNFRSF6-targetting segment wherein the nucleotide sequence of the generic region is set forth in SEQ ID NO:17. In a further embodiment of the invention, the nucleotide sequence of the generic segment of a first isolated nucleic acid molecule is set forth in SEQ ID NO:17 and the sequence of the adjacent TNFRSF6-targetting segment is set forth in SEQ ID NO:1, 3, 5, 7, 9, 11, 13, or 15, or fragments or variants thereof. In an embodiment of the invention an oligonucleotide pair of the invention comprises a second isolated nucleic acid molecule consisting of a generic segment adjacent to a TNFRSF6-targetting segment wherein the nucleotide sequence of the generic region is set forth in SEQ ID NO:18. In a further embodiment of the invention, the nucleotide sequence of the generic segment of a second isolated nucleic acid molecule is set forth in SEQ ID NO:18 and the sequence of the adjacent TNFRSF6-targetting segment is set forth in SEQ ID NO:2, 4, 6, 8, 10, 12, 14, or 16, or fragments or variants thereof.

By “oligonucleotide pair library” is intended a molecular library consisting of a plurality of oligonucleotide pairs of predetermined sequence. By “TNFRSF6 oligonucleotide pair library” is intended an oligonucleotide pair library consisting of oligonucleotide pairs capable of amplifying a region of the TNFRSF6 gene set forth in SEQ ID NO:19.

By “amplification” is intended an increase in the amount of nucleic acid molecules in a sample. Amplifying DNA increases the amount of acid precipitable nucleic acid molecules. The amount of acid precipitable material increases by 1%, 5%, 10%, 15%, 20%, 25%, 30%, 35%, 40%, 45%, 50%, 55%, 60%, 65%, 70%, 75%, 80%, 85%, 90%, 95%, 100%, 150%, 200%, 250%, or more. Methods of quantifying nucleic acid molecules are known in the art and include, but are not limited to, UV absorption spectra, radiolabel incorporation, agarose gel electrophoresis, and ethidium bromide staining. See, for example, Ausubel et al., eds. (2003)Current Protocols in Molecular Biology, (John Wiley & Sons, New York) and Sambrook et al. (2001) Molecular Cloning: A Laboratory Manual (3d ed., Cold Spring Harbor Laboratory Press, Plainview, N.Y., herein incorporated by reference.

Enzymatic amplification is the process of using enzymes to perform amplification. A common type of enzymatic amplification is the polymerase chain reaction (PCR). Known methods of PCR include, but are not limited to, methods using paired primers, nested primers, single specific primers, degenerate primers, gene-specific primers, vector-specific primers, partially-mismatched primers, and the like. Known methods of PCR include, but are not limited to, methods using DNA polymerases from extremophiles and engineered DNA polymerases. It is recognized that it is preferable to use high fidelity PCR reaction conditions in the methods of the invention. See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York).

Methods of performing enzymatic amplification, particularly PCR, are known in the art and discussed elsewhere herein. Known PCR methods include but are not limited to a series of incubation periods or cycles at predetermined temperatures. Various methods of controlling the duration and temperature of each incubation period are known in the art and include, but are not limited to, the use of thermocyclers and transfer between baths at predetermined temperatures. It is envisioned that any means of controlling the PCR reaction incubation conditions may be used in the practice of the invention. In an embodiment a thermocycler is used to regulate the PCR incubation conditions of the enzymatic amplification. By “incubation conditions” is intended the temperature and duration of each incubation period in the enzymatic amplification. In an embodiment the invention provides oligonucleotide pairs that will amplify multiple regions of the TNFRSF6 gene in multiple reactions that are performed under similar incubation conditions. By using similar incubation conditions multiple enzymatic amplification reactions can be performed in one thermocycle chamber simultaneously. The invention reduces the thermocycler resources required for enzymatic amplification of the TNFRSF6 gene. Further, by amplifying the exons and exon-intron boundaries of TNFRSF6 with only eight oligonucleotide pairs the compositions and methods of the invention facilitate automated amplification and sequencing of the gene.

In PCR, typically multiple cycles of a series of incubation periods are performed. Often a single incubation period precedes the cycled incubation period series. The cycle incubation conditions of the incubation period series are designed to facilitate denaturation of the template DNA, annealing of the primer to the template DNA, and extension of the template bound primer. Various factors such as, but not limited to, the primer nucleotide sequence, the template nucleotide sequence, and the type of DNA polymerase can be used to predict suitable temperatures and durations of the incubation periods. An example of incubation conditions suitable for use with the oligonucleotide pairs of the invention is described elsewhere herein. It is recognized that multiple suitable incubation temperatures and durations exist and that the invention is not limited to the precise incubation descriptions described elsewhere herein, particularly with regards to the denaturing incubation temperature and the extension incubation temperature.

The compositions of the invention allow for simultaneous amplification of all TNFRSF6 exons and exon-intron boundaries. The simultaneous amplification requires a carefully controlled temperature for the annealing incubation. The temperature for the annealing incubations of the methods of the invention ranges from 54° C. to 57.9° C., particularly 55° C. to 57.9° C., more particularly 56° C. to 57.5° C., yet more particularly 56.5° C. to 57.5° C. It is recognized that an annealing incubation temperature of 57° C. provides adequate yields of all eight amplicons. It is further recognized that as the primary annealing temperature is varied from 57° C., the ability of the methods of the invention to amplify all TNFRSF6 exons and exon-intron boundaries diminishes. It is also recognized that transitions from the denaturing incubation temperature to the annealing incubation temperature or from the annealing incubation temperature to the extension incubation temperature are not instantaneous and that the enzymatic amplification reaction mixture will pass through other temperature ranges as the incubation temperature shifts from the denaturing incubation temperature to the annealing incubation temperature and from the annealing incubation temperature to the extension temperature or to the storage temperature.

The compositions of the invention allow for simultaneous amplification of all TNFRSF6 exons and exon-intron boundaries in eight amplification reactions that can be performed simultaneously in the same incubation chamber or thermocycler. The methods and compositions of the invention were designed so that eight or fewer amplification reactions can be performed simultaneously. The use of eight or fewer oligonucleotide pairs and thus eight or fewer amplification reactions facilitates high through-put screening, automation, and even robotic use as many of the commercially available reagents such as, but not limited to, plasticware, thermocyclers, multiple pipettors, and robots are currently optimized to work with sets comprising a multiple of eight. These commercially available tools include but are not limited to, 8 well plastic tube strips, 96-well plastic reaction trays, 8-, 16-, 24-, 48-, or 96-well thermocycler devices, 96 well formats for PCR product purification and other molecular biology products. The use of this format also facilitates automated sequencing.

The human TNFRSF6 gene is located on the long arm of chromosome 10 at q24.1. The human genomic sequence (SEQ ID NO:19) spans 28339 nucleotides in 9 exons. The TNFRSF6 family of proteins appears to be involved in Fas mediated apoptosis. Mutations in the TNFRSF6 gene result in ALPS which is characterized by chronic non-malignant lymphadenopathy, splenomegaly, elevated αβCD4⁻-CD8⁻ T cell levels and defective Fas-mediated apoptosis. Considerable clinical variation exists among ALPS patients, and mutation detection is considered essential for accurate diagnosis and prognostic determinations. The oligonucleotide pairs of the invention allow amplification of the exons and exon-intron boundaries of the TNFRSF6 gene.

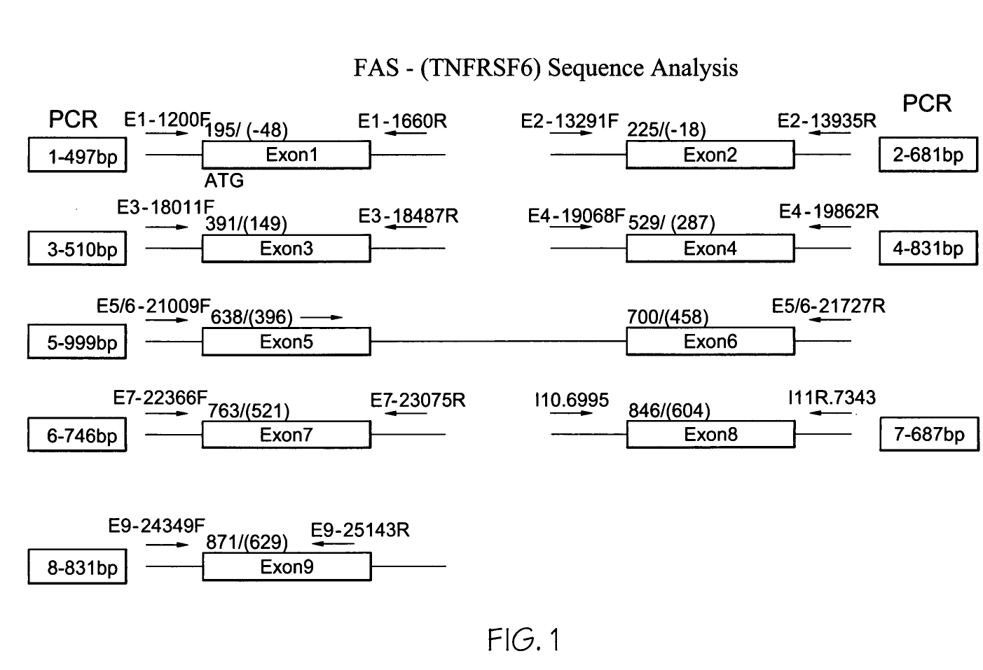

By “region” is intended a portion of the entire nucleotide sequence of interest, such as the genomic TNFRSF6 gene. A region or fragment of the entire nucleotide sequence of interest, such as the genomic TNFRSF6 gene may range from 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 349, 350, 360, 370, 380, 390, 400, 410, 420, 421, 430, 440, 450, 460, 470, 480, 490, 500, 510, 520, 530, 540, 550, 560, 570, 580, 590, 600, 604, 610, 620, 630, 640, 650, 660, 670, 680, 690, 698, 700, 710, 720, 730, 740, 750, 760, 770, 780, 790, 800, 810, 820, 830, 834, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1100, 1150, 1200, 1250, 1300, 1350, 1400, 1450, 1500, 1550, 1600, 1611, 1650, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 10000, 10100, 10200, 10300, 10400, 10500, 10600, 10700, 10800, 10900, 11000, 12000, 13000, 14000, 15000, 16000, 17000, 18000, 19000, 20000, 20100, 20200, 20300, 20400, 20500, 20600, 20700, 20800, 20900, 21000, 21100, 21200, 21300, 21400, 21500, 21600, 21700, 21800, 21900, 22000, 22100, 22200, 22300, 22400, 22500, 22600, 22700, 22800, 22900, 23000, 24000, 24500, 25000, 25500, 26000, 26500, 27000, 27500, 28000, 28, 339 nucleotides, or up to the total number of nucleotides on chromosome 10 q24.1. Such a region contains nucleotide sequence from one or more TNFRSF6 gene exons, one or more TNFRSF6 gene introns, or the regulatory control region operably linked to the TNFRSF6 gene. The oligonucleotide pairs of the invention amplify various regions of the TNFRSF6 gene (See FIG. 1). The oligonucleotide pair comprising a first and second TNFRSF6 targeting segments having the nucleotide sequences set forth in SEQ ID NOS:1 and 2 allows amplification of a region of the TNFRSF6 gene comprising exon 1. The oligonucleotide pair comprising a first and second TNFRSF6 targeting segments having the nucleotide sequences set forth in SEQ ID NOS:3 and 4 allows amplification of a region of the TNFRSF6 gene comprising exon 2. The oligonucleotide pair comprising a first and second TNFRSF6 targeting segments having the nucleotide sequences set forth in SEQ ID NOS:5 and 6 allows amplification of a region of the TNFRSF6 gene comprising exon 3. The oligonucleotide pair comprising a first and second TNFRSF6 targeting segments having the nucleotide sequences set forth in SEQ ID NOS:7 and 8 allows amplification of a region of the TNFRSF6 gene comprising exon 4. The oligonucleotide pair comprising a first and second TNFRSF6 targeting segments having the nucleotide sequences set forth in SEQ ID NOS:9 and 10 allows amplification of a region of the TNFRSF6 gene comprising exons 5-6. The oligonucleotide pair comprising a first and second TNFRSF6 targeting segments having the nucleotide sequences set forth in SEQ ID NOS:11 and 12 allows amplification of a region of the TNFRSF6 gene comprising exon 7. The oligonucleotide pair comprising a first and second TNFRSF6 targeting segments having the nucleotide sequences set forth in SEQ ID NOS:13 and 14 allows amplification of a region of the TNFRSF6 gene comprising exon 8. The oligonucleotide pair comprising a first and second TNFRSF6 targeting segments having the nucleotide sequences set forth in SEQ ID NOS:15 and 16 allows amplification of a region of the TNFRSF6 gene comprising exon 9.

By “biological sample” is intended a sample collected from a subject including, but not limited to, tissues, cells, mucosa, fluid, scrapings, hairs, cell lysates, and secretions. Biological samples such as blood samples can be obtained by any method known to one skilled in the art.

The invention further provides methods of determining the nucleotide sequence of a region or regions of the TNFRSF6 gene. The method involves obtaining a biological sample from a human subject and performing enzymatic amplification of a region of the TNFRSF6 gene using at least one oligonucleotide pair of the invention, providing amplified DNA of a region of the TNFRSF6 gene, and determining the nucleotide sequence of the amplified DNA. It is recognized that an embodiment of the invention involves obtaining a biological sample from a human subject, performing enzymatic amplification of a region of the TNFRSF6 gene, providing amplified DNA of a region of the TNFRSF6 gene, and using microarray sequence analysis to determine the nucleotide sequence of a region or regions of the TNFRSF6 gene.

By “amplified DNA” is intended the product of enzymatic amplification.

Any method of DNA sequencing known in the art can be used in the methods of the invention. Methods of sequencing DNA are known in the art and are described in Graham & Hill eds. (2001) DNA Sequencing Protocols (Humana Press, Totowa N.J.), Kieleczawa ed (2004) DNA Sequencing: Optimizing the Process and Analysis (Jones & Bartlett Publishers, Ontario), Ausubel et al., eds. (2003) Current Protocols in Molecular Biology, (John Wiley & Sons, New York) and Sambrook et al. (2001) Molecular Cloning: A Laboratory Manual (3d ed., Cold Spring Harbor Laboratory Press, Plainview, N.Y., herein incorporated by reference in their entirety). Any method of DNA sequencing known in the art that utilizes a resequencing microarray can be used in the methods of the invention. Methods of microarray sequencing DNA are known in the art and are described in Warrington et al. (2002) Hum Mutat 19:402-409 and Cutler et al. (2001) Genome Res 11: 1913-1925, herein incorporated by reference in their entirety.

A method of the invention involves the step of comparing the nucleotide sequence of a region of a subject's TNFRSF6 gene with a standard sequence profile. Such comparison may be performed by any means known in the art including, but not limited to, manually, electronically, and automatically. It is recognized that the comparison may be performed by a person or machine.

By “standard sequence profile” is intended a listing of the consensus nucleotide sequence of at least one nucleotide sequence of interest. A standard sequence profile may contain additional information including but not limited to, a correlation of at least one nucleotide sequence variation with a disease, syndrome, prognosis, or treatment protocol. A standard sequence profile may exist in printed or electronic form, a remotely accessible form, in a database, or in any other means known in the art. In an embodiment a standard sequence profile is a TNFRSF6 standard sequence profile. For instance, an exemplary TNFRSF6 standard sequence profile correlates certain nucleotide alterations in the TNFRSF6 gene with a TNFRSF6 related syndrome.

In an embodiment, the invention provides kits for performing the methods of the invention. Such kits comprise an isolated nucleic acid molecule of the invention, particularly multiple isolated nucleic acid molecules of the invention, and an oligonucleotide pair of the invention, particularly multiple oligonucleotide pairs of the invention, yet more particularly eight oligonucleotide pairs of the invention. A kit of the invention may further comprise a description of an enzymatic amplification protocol suitable for use with an oligonucleotide pair of the invention, particularly a description of an enzymatic amplification protocol suitable for use with multiple oligonucleotide pairs of the invention, more particularly a description of an enzymatic amplification protocol suitable for use with eight oligonucleotide pairs of the invention. Additionally a kit of the invention may comprise a listing of the consensus TNFRSF6 gene nucleotide sequence in paper or electronic form or a means of accessing a consensus TNFRSF6 gene nucleotide sequence. A kit of the invention may comprise a resequencing microarray chip comprising segments of the TNFRSF6 gene nucleotide sequence.

A kit of the invention may comprise an enzymatic amplification reaction component. By “reaction component” is intended any substance that facilitates the indicated reaction. Reaction components that facilitate the reaction may or may not participate in the chemical processes of the reaction. Reaction components include, but are not limited to, vessels, such as microfuge tubes and multiwell plates; measuring devices, such as micropipette tips and capillary tubes; filters; separation devices such as microfuge tube filter inserts, vacuum apparati, purification resins, magnetic beads, and columns; reagents; compounds; solutions; molecules; buffers; inhibitors; chelating agents; ions; terminators; stabilizers; precipitants; solubilizers; acids; bases; salts; reducing agents; oxidizing agents; enzymes; catalysts; and denaturants. In an embodiment of the invention, concentrated reaction components are provided in kits of the invention. The concentration of the reaction components provided in a kit of the invention may be 1, 2, 3, 4, 5, 6, 7, 8, 9, 10, 15, 20, 25, 30, 35, 40, 50, 60, 70, 80, 90, 100, or more fold concentrated than the desired concentration of the component in the reaction. Reagents and reaction level concentrations of various reagents are discussed elsewhere herein. In an embodiment a kit of the invention may comprise eight enzymatic amplification reaction mixtures each containing an oligonucleotide pair of the invention for use with a subject's DNA.

The following examples are offered by way of illustration and not limitation.

### EXPERIMENTAL

### Example 1

### Extraction of DNA from Whole Blood

Puregene DNA isolation kit (Gentra Systems, Minneapolis, Minn., USA) was used for genomic DNA isolation from blood and other tissues. All reagents were provided by the kit. Whole blood was collected from a patient. The blood was collected in EDTA tubes to prevent the blood from clotting and to reduce DNA degradation. Fresh blood samples were stored at 2-8° C. for up to 7 days. In some instances whole blood was frozen immediately and stored frozen until use.

Enucleated red blood cells were removed from the whole blood sample by lysis. Red blood cell lysis solution (9 ml) was placed in a 15 ml tube. Two to three ml of whole blood were added to the red blood cell lysis solution. The tube contents were mixed by inversion and incubated for 10 minutes at room temperature. The tube was centrifuged for 10 minutes at 2,400 rpm.

The supernatant was removed leaving a visible white pellet containing white blood cells and residual fluid. The pellet was resuspended in the residual fluid by vigorous vortexing.

Cell lysis solution (3 ml) was added to the resuspended cells. The tube was inverted 15-20 times. When cell clumps remained, the tubes were placed in a 37° C. water bath overnight. If cell clumps persisted, 15 μl proteinase K was added and the tube was incubated at 55° C. for one hour to overnight. Samples were stored in cell lysis solution for up to 18 months at room temperature.

Proteins were precipitated by the addition of protein precipitation solution (1 ml). After addition of the protein precipitation solution, the solutions were mixed by vortexing. Proteins were pelleted by centrifugation at 2,400 rpm for 10 minutes. The supernatant (containing the DNA) was transferred into a 15 ml tube containing 3 ml of isopropanol. The solutions were mixed by gentle inversion approximately 50 times. The tubes were spun at 2,400 r.p.m. for three minutes. The pellet size was observed and used to determine the appropriate amount of DNA hydration solution.

The supernatant was removed and the pellet was washed with 3 ml of 70% ethanol. The tubes were spun at 2,400 r.p.m. for 1 minute. The ethanol was removed. The pellet was resuspended in 1 ml 70% ethanol and transferred to a microfuge tube. The microfuge tube was centrifuged for 2 minutes. The supernatant was removed and the pellet was allowed to air dry for 10 minutes.

DNA Hydration Solution was added to the DNA pellet. The DNA pellets were rehydrated overnight at room temperature or by incubation at 65° C. for one hour. The resuspended DNA was stored at 4° C. DNA concentrations were determined by spectrophotometric analysis of a diluted aliquot of the purified DNA.

### Example 2

### Oligonucleotide Synthesis

Oligonucleotides having a generic segment set forth in either SEQ ID NO:17 or SEQ ID NO:18 and a TNFRSF6 targeting segment having the nucleotide sequences set forth in SEQ ID NOS:1-16 were designed. The oligonucleotides were synthesized by a commercial source. The synthetic oligonucleotides were resuspended at a concentration of 100 μM.

### Example 3

### Enzymatic Amplification of Multiple Regions of the TNFRSF6 Gene

PCR amplification is a process sensitive to contamination. Therefore PCR appropriate procedures were used to minimize the risks of contamination.

Master mixes for the PCR reactions were prepared. Master mixes contained 1.5 mM MgCl₂, 1 X PCR buffer (Life Technologies Inc. Rockville, Md., USA), 0.2 mM dNTPs, 1.5 units Taq polymerase, 0.4 μM 5′ primer, and 0.4 μM 3′, primer. The master mix was prepared for the desired number of reactions plus two. Separate master mixes for each oligonucleotide pair were prepared.

PCR tubes were placed on ice. Aliquots of the master mix were transferred into the PCR tubes. Template DNA at 0.5 μg/reaction was added to each tube. The reaction tubes were placed in a thermocycler and the power was turned on. The PCR incubation conditions for the incubation periods were: 95° C., for 5 minutes; 35 cycles of 95° C., for 45 seconds, 57° C., for 45 seconds, and 72° C. for 1 minute, 45 seconds; and one incubation at 72° C. for 10 minutes. Following the 10 minute incubation at 72° C., the reactions were stored at 4° C. PCR product quality was assessed by agarose gel electrophoresis of an aliquot of the PCR reaction. All eight oligonucleotide pairs anneal at 57° C.

The amplified products of the PCR reaction were purified using the Qiaquick PCR Purification kit.

### Example 4

### Preparation of Amplified DNA Samples for Commercial Sequencing

Purified amplified DNA was obtained using the above described processes. The M13F (SEQ ID NO:17) and M13R (SEQ ID NO:18) universal sequencing primers were diluted to 3.3 μM.

Sequencing reaction mixtures were prepared. Each sequencing reaction contained 7 μl distilled, deionized H₂O. Two μl of the appropriate primer was added to each sequencing reaction tube. Three μl of purified PCR product obtained according to the methods described elsewhere above were added to each sequencing reaction tube. The samples were sequenced by a sequencing core facility (CHMC Sequencing Core). When the sequence data was obtained, the sequence information was compared with the TNFRSF6 gene consensus nucleotide sequence. The TNFRSF6 gene consensus nucleotide sequence (SEQ ID NO:19) is available in the NCBI database (AY450925) as is the human Fas antigen nucleotide sequence (SEQ ID NO:20; M67454). Discrepancies between the patient's nucleotide sequence and the consensus sequence of the TNFRSF6 gene indicate a mutation.

All publications, patents, and patent applications mentioned in the specification are indicative of the level of those skilled in the art to which this invention pertains. All publications, patents, and patent applications are herein incorporated by reference to the same extent as if each individual publication or patent application were specifically and individually incorporated by reference.

Having described the invention with reference to the exemplary embodiments, it is to be understood that it is not intended that any limitations or elements describing the exemplary embodiment set forth herein are to be incorporated into the meanings of the patent claims unless such limitations or elements are explicitly listed in the claims. Likewise, it is to be understood that it is not necessary to meet any or all of the identified advantages or objects of the invention disclosed herein in order to fall within the scope of any claims, since the invention is defined by the claims and since inherent and/or unforeseen advantages of the present invention may exist even though they may not be explicitly discussed herein.

Although the foregoing invention has been described in some detail by way of illustration and example for purposes of clarity of understanding, it will be obvious that certain changes and modifications may be practiced within the scope of the appended claims.

That which is claimed:

1. An isolated nucleic acid molecule consisting of a nucleotide sequence selected from the group consisting of:
(a) the nucleotide sequence set forth in SEQ ID NO:1, 2, 3, 4, 5, 6, 7, 8, 10, 11, 12, 13, 14, 15, or 16;
(b) a nucleotide sequence that differs by one nucleotide alteration from that set forth in SEQ ID NO:1, 2, 3, 4, 5, 6, 7, 8, 10, 11, 12, 13, 14, 15, or 16; and
(c) a nucleotide sequence that hybridizes under stringent conditions to a complement of SEQ ID NO:1, 2, 3, 4, 5, 7, 8, 10, 11, 12, 13, 14, or 15.

2. An isolated nucleic acid molecule consisting of a generic segment adjacent to a TNFRSF6-targeting segment wherein said TNFRSF6-targeting segment is at the 3′ end of the molecule and wherein the nucleotide sequence of said TNFRSF6-targeting segment is selected from the group consisting of:
(a) the nucleotide sequence set forth in SEQ ID NO:1, 2, 3, 4, 5, 6, 7, 8, 10, 11, 12, 13, 14, 15, or 16;
(b) a nucleotide sequence that differs by one nucleotide alteration from that set forth in SEQ ID NO:1, 2, 3, 4, 5, 6, 7, 8, 10, 11, 12, 13, 14, 15, or 16; and
(c) a nucleotide sequence that hybridizes under stringent conditions to a complement of SEQ ID NO:1, 2, 3, 4, 5, 7, 8, 10, 11, 12, 13, 14, or 15.

3. The isolated nucleic acid molecule of claim 2, wherein the nucleotide sequence of said generic segment is selected from the group consisting of the nucleotide sequence set forth in SEQ ID NO:17 or SEQ ID NO:18.

4. The isolated nucleic acid molecule of claim 2, wherein the generic segment consists of less than 51 nucleotides.

5. The isolated nucleic acid molecule of claim 2, wherein the nucleotide sequence of said generic segment is set forth in SEQ ID NO:17 and wherein the nucleotide sequence of said TNFRSF6-targeting segment is selected from the group consisting of:
(a) the nucleotide sequence set forth in SEQ ID NO:1, 3, 5, 7, 11, 13, or 15;
(b) a nucleotide sequence that differs by one nucleotide alteration from that set forth in SEQ ID NO: 1, 3, 5, 7, 11, 13, or 15; and
(c) a nucleotide sequence that hybridizes under stringent conditions to a complement of SEQ ID NO: 1, 3, 5, 7, 11, 13, or 15.

6. The isolated nucleic acid molecule of claim 2, wherein the nucleotide sequence of said generic segment is set forth in SEQ ID NO:18 and wherein the nucleotide sequence of said TNFRSF6-targeting segment is selected from the group consisting of:
(a) the nucleotide sequence set forth in SEQ ID NO:2, 4, 6, 8, 10, 12, 14, or 16;
(b) a nucleotide sequence that differs by one nucleotide alteration from that set forth in SEQ ID NO: 2, 4, 6, 8, 10, 12, 14, or 16; and
(c) a nucleotide sequence that hybridizes under stringent conditions to a complement of SEQ ID NO: 2, 4, 8, 10, 12, or 14.

7. The isolated nucleic acid molecule of claim 2, wherein the nucleotide sequence of said generic segment is that of a universal sequencing primer.

8. An oligonucleotide pair comprising a first nucleic acid molecule and a second nucleic acid molecule wherein said first and second nucleic acid molecules allow amplification of a region of the TNFRSF6 gene, said first nucleic acid molecule consisting of a first generic segment adjacent to a first TNFRSF6-targeting segment wherein said first TNFRSF6-targeting segment is at the 3′ end of the molecule and the nucleotide sequence of said first TNFRSF6-targeting segment is selected from the group consisting of:
(a) the nucleotide sequence set forth in SEQ ID NO:1, 3, 5, 7, 9, 11, 13, or 15;
(b) a nucleotide sequence that differs by one nucleotide alteration from that set forth in SEQ ID NO:1, 3, 5, 7, 9, 11, 13, or 15; and
(c) a nucleotide sequence that hybridizes under stringent conditions to a complement of SEQ ID NO:1, 3, 5, 7, 9, 11, 13, or 15; and
said second nucleic acid molecule consisting of a second generic segment adjacent to a second TNFRSF6-targeting segment wherein said second TNFRSF6-targeting segment is at the 3′ end of the molecule and the nucleotide sequence of said second TNFRSF6-targeting segment is selected from the group consisting of:
(a) the nucleotide sequence set forth in SEQ ID NO:2, 4, 6, 8, 10, 12, 14, or 16;
(b) a nucleotide sequence that differs by one nucleotide alteration from that set forth in SEQ ID NO:2, 4, 6, 8, 10, 12, 14, or 16; and
(c) a nucleotide sequence that hybridizes under stringent conditions to a complement of SEQ ID NO:2, 4, 6, 8, 10, 12, 14, or 16.

9. The oligonucleotide pair of claim 8, wherein the nucleotide sequence of said first TNFRSF6-targeting segment is selected from the group consisting of:
(a) the nucleotide sequence set forth in SEQ ID NO:1;
(b) a nucleotide sequence that differs by one nucleotide alteration from that set forth in SEQ ID NO:1; and
(c) a nucleotide sequence that hybridizes under stringent conditions to a complement of SEQ ID NO:1; and
wherein the nucleotide sequence of said second TNFRSF6-targeting segment is selected from the group consisting of:
(a) the nucleotide sequence set forth in SEQ ID NO:2;
(b) a nucleotide sequence that differs by one nucleotide alteration from that set forth in SEQ ID NO:2; and
(c) a nucleotide sequence that hybridizes under stringent conditions to a complement of SEQ ID NO:2.

10. The oligonucleotide pair of claim 8, wherein the nucleotide sequence of said first TNFRSF6-targeting segment is selected from the group consisting of:
(a) the nucleotide sequence set forth in SEQ ID NO:3;
(b) a nucleotide sequence that differs by one nucleotide alteration from that set forth in SEQ ID NO:3; and
(c) a nucleotide sequence that hybridizes under stringent conditions to a complement of SEQ ID NO:3; and
wherein the nucleotide sequence of said second TNFRSF6-targeting segment is selected from the group consisting of:
(a) the nucleotide sequence set forth in SEQ ID NO:4;
(b) a nucleotide sequence that differs by one nucleotide alteration from that set forth in SEQ ID NO:4; and
(c) a nucleotide sequence that hybridizes under stringent conditions to a complement of SEQ ID NO:4.

11. The oligonucleotide pair of claim 8, wherein the nucleotide sequence of said first TNFRSF6-targeting segment is selected from the group consisting of:
(a) the nucleotide sequence set forth in SEQ ID NO:5;
(b) a nucleotide sequence that differs by one nucleotide alteration from that set forth in SEQ ID NO:5; and
(c) a nucleotide sequence that hybridizes under stringent conditions to a complement of SEQ ID NO:5; and
wherein the nucleotide sequence of said second TNFRSF6-targeting segment is selected from the group consisting of:
(a) the nucleotide sequence set forth in SEQ ID NO:6; and
(b) a nucleotide sequence that differs by one nucleotide alteration from that set forth in SEQ ID NO:6; and
(c) a nucleotide sequence that hybridizes under stringent conditions to a complement of SEQ ID NO:6.

12. The oligonucleotide pair of claim 8, wherein the nucleotide sequence of said first TNFRSF6-targeting segment is selected from the group consisting of:
(a) the nucleotide sequence set forth in SEQ ID NO:7;
(b) a nucleotide sequence that differs by one nucleotide alteration from that set forth in SEQ ID NO:7; and
(c) a nucleotide sequence that hybridizes under stringent conditions to a complement of SEQ ID NO:7; and
wherein the nucleotide sequence of said second TNFRSF6-targeting segment is selected from the group consisting of:
(a) the nucleotide sequence set forth in SEQ ID NO:8;
(b) a nucleotide sequence that differs by one nucleotide alteration from that set forth in SEQ ID NO:8; and
(c) a nucleotide sequence that hybridizes under stringent conditions to a complement of SEQ ID NO:8.

13. The oligonucleotide pair of claim 8, wherein the nucleotide sequence of said first TNFRSF6-targeting segment is set forth in SEQ ID NO:9, and wherein the nucleotide sequence of said second TNFRSF6-targeting segment is selected from the group consisting of:
(a) the nucleotide sequence set forth in SEQ ID NO:10;
(b) a nucleotide sequence that differs by one nucleotide alteration from that set forth in SEQ ID NO:10; and
(c) a nucleotide sequence that hybridizes under stringent conditions to a complement of SEQ ID NO:10.

14. The oligonucleotide pair of claim 8, wherein the nucleotide sequence of said first TNFRSF6-targeting segment is selected from the group consisting of:
(a) the nucleotide sequence set forth in SEQ ID NO:11;
(b) a nucleotide sequence that differs by one nucleotide alteration from that set forth in SEQ ID NO:1; and
(c) a nucleotide sequence that hybridizes under stringent conditions to a complement of SEQ ID NO:11; and
wherein the nucleotide sequence of said second TNFRSF6-targeting segment is selected from the group consisting of:
(a) the nucleotide sequence set forth in SEQ ID NO:12;
(b) a nucleotide sequence that differs by one nucleotide alteration from that set forth in SEQ ID NO:12; and
(c) a nucleotide sequence that hybridizes under stringent conditions to a complement of SEQ ID NO:12.

15. The oligonucleotide pair of claim 8, wherein the nucleotide sequence of said first TNFRSF6-targeting segment is selected from the group consisting of:
(a) the nucleotide sequence set forth in SEQ ID NO:13;
(b) a nucleotide sequence that differs by one nucleotide alteration from that set forth in SEQ ID NO:13; and
(c) a nucleotide sequence that hybridizes under stringent conditions to a complement of SEQ ID NO:13; and
wherein the nucleotide sequence of said second TNFRSF6-targeting segment is selected from the group consisting of:
(a) the nucleotide sequence set forth in SEQ ID NO:14;
(b) a nucleotide sequence that differs by one nucleotide alteration from that set forth in SEQ ID NO:14; and
(c) a nucleotide sequence that hybridizes under stringent conditions to a complement of SEQ ID NO:14.

16. The oligonucleotide pair of claim 8, wherein the nucleotide sequence of said first TNFRSF6-targeting segment is selected from the group consisting of:
(a) the nucleotide sequence set forth in SEQ ID NO:15;
(b) a nucleotide sequence that differs by one nucleotide alteration from that set forth in SEQ ID NO:15; and
(c) a nucleotide sequence that hybridizes under stringent conditions to a complement of SEQ ID NO:15; and
wherein the nucleotide sequence of said second TNFRSF6-targeting segment is selected from the group consisting of:
(a) the nucleotide sequence set forth in SEQ ID NO:16;
(b) a nucleotide sequence that differs by one nucleotide alteration from that set forth in SEQ ID NO:16; and
(c) a nucleotide sequence that hybridizes under stringent conditions to a complement of SEQ ID NO:16.

17. A TNFRFSF6 oligonucleotide pair library comprising at least one oligonucleotide pair, said oligonucleotide pair comprising a first nucleic acid molecule and a second nucleic acid molecule wherein said first and second nucleic acid molecules allow amplification of a region of the TNFRSF6 gene, said first nucleic acid molecule consisting of a first generic segment adjacent to a first TNFRSF6-targeting segment wherein said first TNFRSF6-targeting segment is at the 3′ end of the molecule and the nucleotide sequence of said first TNFRSF6-targeting segment is selected from the group consisting of:
(a) the nucleotide sequence set forth in SEQ ID NO:1, 3, 5, 7, 9, 11, 13, or 15;
(b) a nucleotide sequence that differs by one nucleotide alteration from that set forth in SEQ ID NO:1, 3, 5, 7, 9, 11, 13, or 15; and
(c) a nucleotide sequence that hybridizes under stringent conditions to a complement of SEQ ID NO:1, 3, 5, 7, 9, 11, 13, or 15; and
said second nucleic acid molecule consisting of a second generic segment adjacent to a second TNFRSF6-targeting segment wherein said second TNFRSF6-targeting segment is at the 3′ end of the molecule and the nucleotide sequence of said second TNFRSF6-targeting segment is selected from the group consisting of:
(a) the nucleotide sequence set forth in SEQ ID NO:2, 4, 6, 8, 10, 12, 14, or 16;
(b) a nucleotide sequence that differs by one nucleotide alteration from that set forth in SEQ ID NO:2, 4, 6, 8, 10, 12, 14, or 16; and
(c) a nucleotide sequence that hybridizes under stringent conditions to a complement of SEQ ID NO:2, 4, 6, 8, 10, 12, 14, or 16.

18. The TNFRFSF6 oligonucleotide pair library of claim 17 wherein the oligonucleotide pair library is comprised of oligonucleotide pairs selected from the group consisting of:
(a) an oligonucleotide pair comprising a first nucleic acid molecule comprising a first TNFRSF6-targeting segment having the nucleotide sequence set forth in SEQ ID NO:1 and a second nucleic acid molecule comprising a second TNFRSF6-targeting segment having the nucleotide sequence set forth in SEQ ID NO:2;
(b) an oligonucleotide pair comprising a first nucleic acid molecule comprising a first TNFRSF6-targeting segment having the nucleotide sequence set forth in SEQ ID NO:3 and a second nucleic acid molecule comprising a second TNFRSF6-targeting segment having the nucleotide sequence set forth in SEQ ID NO:4;
(c) an oligonucleotide pair comprising a first nucleic acid molecule comprising a first TNFRSF6-targeting segment having the nucleotide sequence set forth in SEQ ID NO:5 and a second nucleic acid molecule comprising a second TNFRSF6-targeting segment having the nucleotide sequence set forth in SEQ ID NO:6;
(d) an oligonucleotide pair comprising a first nucleic acid molecule comprising a first TNFRSF6-targeting segment having the nucleotide sequence set forth in SEQ ID NO:7 and a second nucleic acid molecule comprising a second TNFRSF6-targeting segment having the nucleotide sequence set forth in SEQ ID NO:8;
(e) an oligonucleotide pair comprising a first nucleic acid molecule comprising a first TNFRSF6-targeting segment having the nucleotide sequence set forth in SEQ ID NO:9 and a second nucleic acid molecule comprising a second TNFRSF6-targeting segment having the nucleotide sequence set forth in SEQ ID NO:10;
(f) an oligonucleotide pair comprising a first nucleic acid molecule comprising a first TNFRSF6-targeting segment having the nucleotide sequence set forth in SEQ ID NO:11 and a second nucleic acid molecule comprising a second TNFRSF6-targeting segment having the nucleotide sequence set forth in SEQ ID NO:12;
(g) an oligonucleotide pair comprising a first nucleic acid molecule comprising a first TNFRSF6-targeting segment having the nucleotide sequence set forth in SEQ ID NO:13 and a second nucleic acid molecule comprising a second TNFRSF6-targeting segment having the nucleotide sequence set forth in SEQ ID NO:14; and
(h) an oligonucleotide pair comprising a first nucleic acid molecule comprising a first TNFRSF6-targeting segment having the nucleotide sequence set forth in SEQ ID NO:15 and a second nucleic acid molecule comprising a second TNFRSF6-targeting segment having the nucleotide sequence set forth in SEQ ID NO:16.

19. A method of amplifying a region of the TNFRSF6 gene comprising the steps of:
(a) obtaining a biological sample from a human subject; and
(b) performing enzymatic amplification using a first oligonucleotide pair selected from the TNFRFSF6 oligonucleotide pair library of claim 17.

20. The method of claim 19, further comprising using a second oligonucleotide pair to perform enzymatic amplification of a second region of the TNFRSF6 gene.

21. The method of claim 20, wherein the incubation conditions of the enzymatic amplification of the first and second regions are identical.

22. The method of claim 20, further comprising using a third oligonucleotide pair to perform enzymatic amplification of a third region of the TNFRSF6 gene.

23. The method of claim 22, further comprising using a fourth oligonucleotide pair to perform enzymatic amplification of a fourth region of the TNFRSF6 gene.

24. The method of claim 23, further comprising using a fifth oligonucleotide pair to perform enzymatic amplification of a fifth region of the TNFRSF6 gene.

25. The method of claim 24, further comprising using a sixth oligonucleotide pair to perform enzymatic amplification of a sixth region of the TNFRSF6 gene.

26. The method of claim 25, further comprising using a seventh oligonucleotide pair to perform enzymatic amplification of a seventh region of the TNFRSF6 gene.

27. The method of claim 26, further comprising using an eighth oligonucleotide pair to perform enzymatic amplification of an eighth region of the TNFRSF6 gene.

28. The method of claim 27, wherein the incubation conditions of the enzymatic amplification of the eight regions are identical.

29. A kit for performing a method of amplifying a region of the TNFRSF6 gene comprising a TNFRFSF6 oligonucleotide pair library comprising at least one oligonucleotide pair, said oligonucleotide pair comprising a first nucleic acid molecule and a second nucleic acid molecule wherein said first and second nucleic acid molecules allow amplification of a region of the TNFRSF6 gene, said first nucleic acid molecule consisting of a first generic segment adjacent to a first TNFRSF6-targeting segment wherein said first TNFRSF6-targeting segment is at the 3′ end of the molecule and the nucleotide sequence of said first TNFRSF6-targeting segment is selected from the group consisting of:
(a) the nucleotide sequence set forth in SEQ ID NO:1, 3, 5, 7, 9, 11, 13, or 15;
(b) a nucleotide sequence that differs by one nucleotide alteration from that set forth in SEQ ID NO:1, 3, 5, 7, 9, 11, 13, or 15; and
(c) a nucleotide sequence that hybridizes under stringent conditions to a complement of SEQ ID NO:1, 3, 5, 7, 9, 11, 13, or 15; and
said second nucleic acid molecule consisting of a second generic segment adjacent to a second TNFRSF6-targeting segment wherein said second TNFRSF6-targeting segment is at the 3′ end of the molecule and the nucleotide sequence of said second TNFRSF6-targeting segment is selected from the group consisting of:
(a) the nucleotide sequence set forth in SEQ ID NO:2, 4, 6, 8, 10, 12, 14, or 16;
(b) a nucleotide sequence that differs by one nucleotide alteration from that set forth in SEQ ID NO:2, 4, 6, 8, 10, 12, 14, or 16; and
(c) a nucleotide sequence that hybridizes under stringent conditions to a complement of SEQ ID NO:2, 4, 6, 8, 10, 12, 14, or 16.

30. The kit of claim 29 wherein the TNFRFSF6 oligonucleotide pair library is comprised of at least one oligonucleotide pair selected from the group consisting of:
(a) an oligonucleotide pair comprising a first nucleic acid molecule comprising a first TNFRSF6-targeting segment having the nucleotide sequence set forth in SEQ ID NO:1 and a second nucleic acid molecule comprising a second TNFRSF6-targeting segment having the nucleotide sequence set forth in SEQ ID NO:2;
(b) an oligonucleotide pair comprising a first nucleic acid molecule comprising a first TNFRSF6-targeting segment having the nucleotide sequence set forth in SEQ ID NO:3 and a second nucleic acid molecule comprising a second TNFRSF6-targeting segment having the nucleotide sequence set forth in SEQ ID NO:4;
(c) an oligonucleotide pair comprising a first nucleic acid molecule comprising a first TNFRSF6-targeting segment having the nucleotide sequence set forth in SEQ ID NO:5 and a second nucleic acid molecule comprising a second TNFRSF6-targeting segment having the nucleotide sequence set forth in SEQ ID NO:6;
(d) an oligonucleotide pair comprising a first nucleic acid molecule comprising a first TNFRSF6-targeting segment having the nucleotide sequence set forth in SEQ ID NO:7 and a second nucleic acid molecule comprising a second TNFRSF6-targeting segment having the nucleotide sequence set forth in SEQ ID NO:8;
(e) an oligonucleotide pair comprising a first nucleic acid molecule comprising a first TNFRSF6-targeting segment having the nucleotide sequence set forth in SEQ ID NO:9 and a second nucleic acid molecule comprising a second TNFRSF6-targeting segment having the nucleotide sequence set forth in SEQ ID NO:10;
(f) an oligonucleotide pair comprising a first nucleic acid molecule comprising a first TNFRSF6-targeting segment having the nucleotide sequence set forth in SEQ ID NO:11 and a second nucleic acid molecule comprising a second TNFRSF6-targeting segment having the nucleotide sequence set forth in SEQ ID NO:12;
(g) an oligonucleotide pair comprising a first nucleic acid molecule comprising a first TNFRSF6-targeting segment having the nucleotide sequence set forth in SEQ ID NO:13 and a second nucleic acid molecule comprising a second TNFRSF6-targeting segment having the nucleotide sequence set forth in SEQ ID NO:14; and
(h) an oligonucleotide pair comprising a first nucleic acid molecule comprising a first TNFRSF6-targeting segment having the nucleotide sequence set forth in SEQ ID NO:15 and a second nucleic acid molecule comprising a second TNFRSF6-targeting segment having the nucleotide sequence set forth in SEQ ID NO:16.

31. A method of determining the nucleotide sequence of a region of the TNFRSF6 gene of a subject comprising the steps of:
(a) obtaining a biological sample from a human subject;
(b) performing enzymatic amplification of a region of the TNFRSF6 gene using an oligonucleotide pair, said oligonucleotide pair comprising a first nucleic acid molecule and a second nucleic acid molecule wherein said first and second nucleic acid molecules allow amplification of a region of the TNFRSF6 gene, said first nucleic acid molecule consisting of a first generic segment adjacent to a first TNFRSF6-targeting segment wherein said first TNFRSF6-targeting segment is at the 3′ end of the molecule and the nucleotide sequence of said first TNFRSF6-targeting segment is selected from the group consisting of:
(i) the nucleotide sequence set forth in SEQ ID NO:1, 3, 5, 7, 9, 11, 13, or 15;
(ii) a nucleotide sequence that differs by one nucleotide alteration from that set forth in SEQ ID NO:1, 3, 5, 7, 9, 11, 13, or 15; and
(iii) a nucleotide sequence that hybridizes under stringent conditions to a complement of SEQ ID NO:1, 3, 5, 7, 9, 11, 13, or 15; and
said second nucleic acid molecule consisting of a second generic segment adjacent to a second TNFRSF6-targeting segment wherein said second TNFRSF6-targeting segment is at the 3′ end of the molecule and the nucleotide sequence of said second TNFRSF6-targeting segment is selected from the group consisting of:
(i) the nucleotide sequence set forth in SEQ ID NO:2, 4, 6, 8, 10, 12, 14, or 16;
(ii) a nucleotide sequence that differs by one nucleotide alteration from that set forth in SEQ ID NO:2, 4, 6, 8, 10, 12, 14, or 16; and
(iii) a nucleotide sequence that hybridizes under stringent conditions to a complement of SEQ ID NO:2, 4, 6, 8, 10, 12, 14, or 16;
(c) providing amplified DNA of a region of the TNFRSF6 gene; and
(d) determining the nucleotide sequence of said amplified DNA.

32. The method of claim 31 wherein at least one universal sequencing primer is used in determining the nucleotide sequence of said amplified DNA.

33. The method of claim 32, wherein the nucleotide sequence of at least one generic region comprises the nucleotide sequence of said universal sequencing primer.

34. The method of claim 32, wherein said oligonucleotide pair is selected from the group consisting of:
(a) an oligonucleotide pair comprising a first nucleic acid molecule comprising a first TNFRSF6-targeting segment having the nucleotide sequence set forth in SEQ ID NO:1 and a second nucleic acid molecule comprising a second TNFRSF6-targeting segment having the nucleotide sequence set forth in SEQ ID NO:2;
(b) an oligonucleotide pair comprising a first nucleic acid molecule comprising a first TNFRSF6-targeting segment having the nucleotide sequence set forth in SEQ ID NO:3 and a second nucleic acid molecule comprising a second TNFRSF6-targeting segment having the nucleotide sequence set forth in SEQ ID NO:4;
(c) an oligonucleotide pair comprising a first nucleic acid molecule comprising a first TNFRSF6-targeting segment having the nucleotide sequence set forth in SEQ ID NO:5 and a second nucleic acid molecule comprising a second TNFRSF6-targeting segment having the nucleotide sequence set forth in SEQ ID NO:6;
(d) an oligonucleotide pair comprising a first nucleic acid molecule comprising a first TNFRSF6-targeting segment having the nucleotide sequence set forth in SEQ ID NO:7 and a second nucleic acid molecule comprising a second TNFRSF6-targeting segment having the nucleotide sequence set forth in SEQ ID NO:8;
(e) an oligonucleotide pair comprising a first nucleic acid molecule comprising a first TNFRSF6-targeting segment having the nucleotide sequence set forth in SEQ ID NO:9 and a second nucleic acid molecule comprising a second TNFRSF6-targeting segment having the nucleotide sequence set forth in SEQ ID NO:10;
(f) an oligonucleotide pair comprising a first nucleic acid molecule comprising a first TNFRSF6-targeting segment having the nucleotide sequence set forth in SEQ ID NO:11 and a second nucleic acid molecule comprising a second TNFRSF6-targeting segment having the nucleotide sequence set forth in SEQ ID NO:12;
(g) an oligonucleotide pair comprising a first nucleic acid molecule comprising a first TNFRSF6-targeting segment having the nucleotide sequence set forth in SEQ ID NO:13 and a second nucleic acid molecule comprising a second TNFRSF6-targeting segment having the nucleotide sequence set forth in SEQ ID NO:14; and
(h) an oligonucleotide pair comprising a first nucleic acid molecule comprising a first TNFRSF6-targeting segment having the nucleotide sequence set forth in SEQ ID NO:15 and a second nucleic acid molecule comprising a second TNFRSF6-targeting segment having the nucleotide sequence set forth in SEQ ID NO:16.

35. A method of diagnosing a TNFRSF6-related syndrome comprising the steps of:
(a) obtaining a biological sample from a human subject;
(b) performing enzymatic amplification of a region of the TNFRSF6 gene using an oligonucleotide pair, said oligonucleotide pair comprising a first nucleic acid molecule and a second nucleic acid molecule wherein said first and second nucleic acid molecules allow amplification of a region of the TNFRSF6 gene, said first nucleic acid molecule consisting of a first generic segment adjacent to a first TNFRSF6-targeting segment wherein said first TNFRSF6-targeting segment is at the 3′ end of the molecule and the nucleotide sequence of said first TNFRSF6-targeting segment is selected from the group consisting of:
(i) the nucleotide sequence set forth in SEQ ID NO:1, 3, 5, 7, 9, 11, 13, or 15;
(ii) a nucleotide sequence that differs by one nucleotide alteration from that set forth in SEQ ID NO:1, 3, 5, 7, 9, 11, 13, or 15; and
(iii) a nucleotide sequence that hybridizes under stringent conditions to a complement of SEQ ID NO:1, 3, 5, 7, 9, 11, 13, or 15; and
said second nucleic acid molecule consisting of a second generic segment adjacent to a second TNFRSF6-targeting segment wherein said second TNFRSF6-targeting segment is at the 3′ end of the molecule and the nucleotide sequence of said second TNFRSF6-targeting segment is selected from the group consisting of:
(i) the nucleotide sequence set forth in SEQ ID NO:2, 4, 6, 8, 10, 12, 14, or 16;
(ii) a nucleotide sequence that differs by one nucleotide alteration from that set forth in SEQ ID NO:2, 4, 6, 8, 10, 12, 14, or 16; and
(iii) a nucleotide sequence that hybridizes under stringent conditions to a complement of SEQ ID NO:2, 4, 6, 8, 10, 12, 14, or 16;
(c) providing amplified DNA of a region of the TNFRSF6 gene;
(d) determining the nucleotide sequence of said amplified DNA; and
(e) comparing said nucleotide sequence of said amplified DNA with a standard sequence profile.

36. The method of claim 35, wherein said TNFRSF6-related syndrome is autoimmune lymphoproliferative syndrome.

37. The method of claim 35, wherein enzymatic amplification of multiple regions of the TNFRSF6 gene occurs.

38. The method of claim 37, wherein simultaneous enzymatic amplification of multiple regions occurs.

39. The method of claim 37, wherein the nucleotide sequences of multiple amplified DNA are determined.

40. The method of claim 39, wherein the nucleotide sequences of the TNFRSF6 gene 5′ upstream regulatory region, exons, and exon-intron boundaries are determined.

41. The method of claim 38, wherein the temperature of the annealing incubation of the enzymatic amplification is selected from the group consisting of:
(a) an annealing temperature of 57° C., and
(b) an annealing temperature within the range of 56° C. to 57.9° C.
